(19) United States
(12) Patent Application Publication
(10) Pub. No.: US 2011/0311650 A1
(43) Pub. Date: Dec. 22, 2011

(54) MULTIPLEXED BIOMARKERS OF INSULIN RESISTANCE

(76) Inventors: Thomas Wang, Lexington, MA (US); Oded Shaham, Cambridge, MA (US); Robert Gerszten, Brookline, MA (US); Vamsi K. Mootha, Cambridge, MA (US); Vasan S. Ramachandran, Berlin, MA (US); Martin Larson, Chestnut Hill, MA (US)

(21) Appl. No.: 13/002,815

(22) PCT Filed: Jul. 7, 2009
(86) PCT No.: PCT/US2009/049831

§ 371 Date: Sep. 8, 2011

Related U.S. Application Data
(60) Provisional application No. 61/134,154, filed on Jul. 7, 2008.

(51) Int. Cl. A61K 38/28; G01N 33/74; A61K 38/26; H01J 49/26; A61K 31/19; A61K 33/24; A61P 3/10; G01N 33/53; C12Q 1/54

(52) U.S. Cl. 424/646; 435/7.92; 436/501; 514/7.2; 435/14; 514/5.9; 514/557; 250/282

(57) ABSTRACT

The invention, in some aspects, relates to methods for characterizing glucose-related metabolic disorders. In some aspects, the invention relates to methods and kits useful for diagnosing, classifying, profiling, and treating glucose-related metabolic disorders. In some aspects, the invention relates to methods useful for diagnosing, classifying, profiling, and treating diabetes.

### CLAIM OF PRIORITY

This application claims the benefit of U.S. Provisional Patent Application Ser. No. 61/134,154, filed on Jul. 7, 2008, the entire contents of which are hereby incorporated by reference.

### GOVERNMENT FUNDING

This invention was made with Government support from the National Institutes of Health under Grant Nos. R01DK081572-02 and MO1-RR01066. The Government has certain rights in the invention.

### FIELD OF THE INVENTION

The invention, in some aspects, relates to methods for determining a subject's risk of developing a glucose-related metabolic disorder, e.g., impaired glucose tolerance or diabetes, e.g., type 2 diabetes. In some aspects, the invention relates to methods and kits useful for diagnosing, classifying, profiling, and treating glucose-related metabolic disorders. In some aspects, the invention relates to methods useful for diagnosing, classifying, profiling, and treating diabetes.

### BACKGROUND OF INVENTION

Glucose homeostasis is a complex physiologic process involving the orchestration of regulatory mechanisms spanning multiple organ systems. During an overnight fast, for instance, glucose levels are maintained through both glycogenolysis and gluconeogenesis. In addition, the central nervous system, a major consumer of glucose, reduces its reliance on glucose by shifting to the use of ketone bodies (e.g., acetoacetate, β-hydroxybutyrate), which are synthesized in the liver from fatty acids released from adipose tissue. Ingestion of glucose after overnight fasting then triggers the rapid release of insulin from the pancreas, which promotes glucose uptake in peripheral tissues. Insulin also causes many metabolic pathways to shift from catabolism to anabolism. For example, proteolysis in skeletal muscle and associated release of alanine and glutamine (which support hepatic gluconeogenesis) are replaced by amino acid uptake and protein synthesis. Also, triacylglycerol lysis in adipose tissue and hepatic synthesis of ketone bodies are inhibited and replaced by fatty acid uptake and re-esterification. Hence the transition from fasting to feeding is accompanied by many changes in metabolite concentration, as the body makes adjustments to achieve glucose homeostasis.

While it is well appreciated that loss of glucose homeostasis and insulin dysfunction are linked with the development of diabetes, the complex relationship between global metabolite concentrations, glucose homeostasis, and diabetes remains minimally understood.

### SUMMARY OF INVENTION

Glucose ingestion after an overnight fast triggers the fasting:feeding transition, an insulin-dependent, homeostatic program altered in diabetes. To systematically characterize the dynamics of this program, a high-performance liquid chromatography with tandem mass spectrometry detection (LC-MS/MS) strategy has been developed to simultaneously measure 191 metabolites in human plasma during the oral glucose tolerance test (OGTT). In two separate cohorts, 18 metabolites changed reproducibly, including bile acids, urea cycle intermediates, and purine degradation products, none of which were previously linked to glucose homeostasis. The metabolite dynamics disclosed herein reflected the action of insulin on proteolysis, lipolysis, ketogenesis, and glycolysis. Profiling subjects with glucose-related metabolic disorders (i.e., prediabetics) indicated that the 2-hour changes in glycerol and leucine/isoleucine jointly provide strong prediction of insulin sensitivity and reveal the individuality of insulin action. For example, in some cases, humans are selectively resistant to insulin's suppression of lipolysis, while others are selectively resistant to proteolysis. The individuality of insulin action is readily detected by the metabolic profiling methods disclosed herein, which provide a useful adjunct to OGTT for classifying and predicting diabetes.

In one aspect, the invention provides methods for diagnosing or determining likelihood (or risk) of developing a glucose related metabolic disorder in a subject. The methods include determining levels or occurrences of a plurality of biomarkers in a clinical sample obtained from the subject, wherein the plurality of biomarkers are selected from an amino acid, a glucose metabolite, a ketone body, a lipid metabolite, and a bile acid, and wherein the levels or the occurrences of the plurality of biomarkers are indicative of the glucose related metabolic disorder in the subject. In some embodiments, the methods further include performing a comparison between the levels or occurrences of the plurality of biomarkers and a reference, wherein the comparison is indicative of whether or not the subject has the glucose related metabolic disorder.

In one aspect, the invention provides methods for determining the risk of developing a glucose related metabolic disorder, e.g., diabetes, in a subject. In some embodiments, the methods determine the subject's risk of developing the disorder within 20 years, within 15 years, within 12 years, within 10 years, within 5 years, or within 1 year. The methods include optionally providing a biological sample from the subject; determining a level of two or more, e.g., three or more, four or more, five or more, six or more, or all seven, metabolic biomarkers in the sample, wherein the metabolic biomarkers are selected from the group consisting of isoleucine, phenylalanine, tyrosine, valine, leucine, tryptophan, and ornithine; and comparing the levels of the metabolic biomarkers with reference levels of the same biomarkers. In some embodiments, the reference levels represent levels of the biomarker in the top (highest) quartile, e.g., a threshold that delimits the lower end of the top quartile, such that a level above the reference level indicates that the subject is in the top quartile for that metabolite. The presence of levels of the metabolic biomarkers that are higher than the reference levels indicates an increased risk of developing diabetes in the subject.

In some embodiments, Add the methods include determining excursions (e.g., ratios or differences) in the biomarkers between two states, e.g., between the fasting and the non-fasting (post-glucose, e.g., during or after an OGTT) state. As demonstrated herein, a excursions are predictive of fasting insulin levels.

In some embodiments, the methods include determining levels of isoleucine and one or more of, e.g., two or more, three or more, four or more, or five or more of, phenylalanine, tyrosine, valine, leucine, tryptophan, or ornithine. In some embodiments, the methods include determining levels of phenylalanine and one or more of, e.g., two or more, three or more, four or more, or five or more of, isoleucine, tyrosine, valine, leucine, tryptophan, or ornithine. In some embodiments, the methods include determining levels of tyrosine and one or more of, e.g., two or more, three or more, four or more, or five or more of, valine, isoleucine, phenylalanine, leucine, tryptophan, or ornithine. In some embodiments, the methods include determining levels of valine and one or more of, e.g., two or more, three or more, four or more, or five or more of, phenylalanine, tyrosine, isoleucine, leucine, tryptophan, or ornithine. In some embodiments, the methods include determining levels of tryptophan and one or more of, e.g., two or more, three or more, four or more, or five or more of, phenylalanine, tyrosine, valine, leucine, isoleucine, or ornithine. In some embodiments, the methods include determining levels of leucine and one or more of, e.g., two or more, three or more, four or more, or five or more of, phenylalanine, tyrosine, valine, isoleucine, tryptophan, or ornithine. In some embodiments, the methods include determining levels of ornithine and one or more of, e.g., two or more, three or more, four or more, or five or more of, phenylalanine, tyrosine, valine, leucine, tryptophan, or isoleucine.

In some embodiments, the methods include determining levels of isoleucine, phenylalanine, tyrosine, valine, leucine, tryptophan, and ornithine.

In some embodiments, the methods further include determining a level of an additional biomarker selected from the group consisting of glycerol, lactate, and β-hydroxybutyrate. In some embodiments, the methods further include determining a level of an additional biomarker selected from the group consisting of lactate, and β-hydroxybutyrate.

In some embodiments, the methods further include determining a level of an additional biomarker selected from the group consisting of citrulline, glycochenodeoxycholic acid, glycocholic acid, hippuric acid, histidine, hypoxanthine, lysine, methionine, pyruvate, and taurochenodeoxycholic acid. In some embodiments, the methods further include assessing one or both of glucose and insulin levels in the subject.

In some embodiments, the subject has normal glucose tolerance, i.e., a glucose tolerance level below 140 mg/dl and normal fasting glucose levels below 100 mg/dl.

In some embodiments, the methods further include selecting a treatment (i.e., a treatment for diabetes) for the subject based on the comparison of the levels of the metabolic biomarkers with the reference levels. In some embodiments, the methods further include administering the selected treatment to the subject. A care giver, e.g., a physician, will readily be able to select an appropriate treatment for the subject. In some embodiments, the treatment is administering to the subject an effective amount of at least one anti-diabetes compound, and/or instructing the subject to adopt at least one lifestyle change.

In some embodiments, the sample is or includes serum, plasma, or blood from the subject. In some embodiments, the biological sample is obtained from the subject following a fast, e.g., a fast for between 6 and 16 hours.

In some embodiments, the subject has at least one traditional risk factor for diabetes, e.g., as described herein.

In some embodiments, the levels of the biomarkers are determined using a mass spectrometer.

In another aspect, the invention provides kits for determining the presence or risk of a glucose related metabolic disorder in a subject. The kits include reagents suitable for determining levels of a plurality of biomarkers in a test sample, wherein the plurality of biomarkers comprises two or more, e.g., three or more, four or more, five or more, six or more, or all seven of isoleucine, phenylalanine, tyrosine, valine, leucine, tryptophan, and ornithine; optionally one or more control samples comprising predetermined levels of the same biomarkers, wherein a comparison of the levels of the biomarkers in the test sample with the levels in the control samples indicates the presence of risk of a glucose related metabolic disorder in the subject; and instructions for use of the kit in a method described herein. The kit can further include containers or substrates for performing a method described herein.

In some embodiments of the methods described herein, the glucose related metabolic disorder is diabetes, impaired fasting glycemia, impaired glucose tolerance, or metabolic syndrome. In some embodiments, the diabetes is selected from: type I diabetes, type II diabetes, gestational diabetes, polycystic ovary syndrome, and another specific type of diabetes. In certain embodiments, the other specific type of diabetes is associated with a genetic defect, a genetic syndrome, a genetically determined abnormality, an exocrine pancreas defect, an endocrinopathy, a drug or chemical cause, an infection, or an immunological pathogenesis different from that which leads to type I diabetes.

In some embodiments of any of the foregoing methods, the bile acid is selected from glycocholic acid, glycochendeoxycholic acid, and taurochenodeoxycholic acid.

In some embodiments of any of the foregoing methods, the plurality of biomarkers comprise an amino acid, a glucose metabolite, a ketone body, and a lipid metabolite.

In some embodiments of any of the foregoing methods, the glucose metabolite is selected from glucose, pyruvate, lactate, and malate.

In some embodiments of any of the foregoing methods, the ketone body is selected from beta-hydroxybutyrate, acetoacetate, and acetone.

In some embodiments of any of the foregoing methods, the plurality of biomarkers comprise an amino acid and a lipid metabolite.

In some embodiments of any of the foregoing methods, the amino acid is isoleucine or leucine.

In some embodiments of any of the foregoing methods, the amino acid is a non-proteinogenic amino acid. In certain embodiments, the non-proteinogenic amino acid is citrulline or ornithine.

In some embodiments of any of the foregoing methods, the amino acid is selected from: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In some embodiments of any of the foregoing methods, the amino acid is a branched chain amino acid.

In some embodiments of any of the foregoing methods, the lipid metabolite is glycerol.

In some embodiments, the methods include determining levels of glycerol and levels of isoleucine and/or leucine in a clinical sample obtained from the subject, wherein the levels of glycerol and the levels of isoleucine and/or leucine are indicative of the presence of, or risk of developing, impaired glucose tolerance. In some embodiments, the methods further include performing a comparison between the levels of glycerol and the levels of isoleucine and/or leucine and a reference, wherein the comparison is indicative of the presence of, or risk of developing, impaired glucose tolerance.

In some embodiments, the methods include determining levels of two or more metabolic biomarkers selected from the group consisting of: isoleucine, phenylalanine, tyrosine, valine, leucine, tryptophan, and ornithine, in a sample from the subject, wherein the levels of glycerol and the levels of isoleucine and/or leucine are indicative of the presence of, or risk of developing, diabetes, e.g., type 2 diabetes. In some embodiments, the methods further include performing a comparison between the levels of the two or more metabolic biomarkers and reference levels of the same biomarkers, wherein the comparison is indicative of the presence of, or risk of developing, diabetes, e.g., type 2 diabetes.

In some embodiments of any of the foregoing methods, the reference represents levels of the plurality of biomarkers in a non-diabetic control. In certain embodiments, the non-diabetic control has a glucose tolerance level below 140 mg/dl and/or normal fasting glucose levels or occurrences below 100 mg/dl. In certain embodiments, a level of a biomarker in a subject that is statistically significantly different than a reference level in a non-diabetic control is indicative of the presence or increased risk of developing impaired glucose tolerance or diabetes in the subject. In certain embodiments, a level of a biomarker in a subject that is not statistically significantly different than, i.e., is statistically similar to, a reference level in a non-diabetic control is indicative of the absence of, or no increased risk (normal risk) of developing impaired glucose tolerance or diabetes in the subject.

In some embodiments of any of the foregoing methods, the reference represents levels of the plurality of biomarkers in a diabetic control. In certain embodiments, the diabetic control has a glucose tolerance level at or above 140 mg/dl (e.g., 140 and 199 mg/dl) and/or normal fasting glucose levels or occurrences at or above 100 mg/dl. In certain embodiments, a level of a biomarker in a subject that is statistically significantly different than a reference level in a diabetic control is indicative of the absence of, or no increased risk (normal risk) of developing impaired glucose tolerance or diabetes in the subject. In certain embodiments, a level of a biomarker in a subject that is not statistically significantly different than, i.e., is statistically similar to, a reference level in a diabetic control is indicative of the presence or increased risk of developing impaired glucose tolerance or diabetes in the subject.

In some embodiments of any of the foregoing methods, the clinical sample is obtained from the subject in conjunction with (e.g., before, during, or after) an oral glucose tolerance test on the subject. In certain embodiments, the oral glucose tolerance test comprises having the subject fast, optionally wherein the fast is for between 6 and 16 hours. In certain embodiments, the methods further include administering a dose of glucose to the subject after the fast, optionally wherein the dose of glucose is between 1.5 to 2 grams of glucose per kilogram of the subject and/or approximately 75 grams of glucose. In certain of these embodiments, the methods further include obtaining the clinical sample at an interval of the glucose tolerance test selected from: before glucose administration and approximately 30, approximately 60, approximately 90, and approximately 120 minutes after glucose administration. In some embodiments of the methods described herein, a sample obtained from the subject before glucose administration is used as a reference sample, and reference levels are determined in that reference sample.

In some embodiments of the methods described herein, the methods can further include assessing glucose and/or insulin levels or occurrences in the subject, optionally wherein the glucose level is determined in a hexokinase assay, and optionally wherein the insulin level is determined using a radio immunoassay. In some embodiments, the methods further include determining weight, hip-waist ratio, or body mass index (BMI), and using the results of that determination in addition to the levels of metabolic biomarkers as described herein to determine a subject's risk of developing a glucose related metabolic disorder, e.g., impaired glucose tolerance or diabetes. For example, the presence of overweight (e.g., BMI of 25-29), or of a waist-to-hip ratio of 0.8-0.85 for women or 0.95-1.0 for men, indicates a moderately increased risk of developing a glucose-related metabolic disorder. The presence of obesity (BMI>29) or of a waist-to-hip ratio of over 0.85 for women or over 1.0 for men, indicates a high risk of developing a glucose-related metabolic disorder.

In some embodiments of the methods described herein, the clinical sample is or comprises serum or plasma. In some embodiments, the levels of the plurality of biomarkers are in the clinical sample.

In some embodiments of any of the foregoing methods, the subject has at least one traditional diabetic risk factor. In certain embodiments, the traditional diabetic risk factor is selected from: greater than 40 years of age, pregnancy, excess body weight, family history of diabetes, low HDL cholesterol (e.g., under 40 mg/dl), high triglyceride levels or occurrences (e.g., 250 mg/dL or more), high blood pressure (e.g., greater than or equal to 140/90 mmHg), impaired glucose tolerance, low activity level, poor diet, and from an ethnic groups selected from African American, Hispanic American, and Native American.

In some embodiments of any of the foregoing methods, the methods include determining levels of the plurality of biomarkers at least twice, e.g., to determine relative levels between two states in the subject. In certain embodiments, the two states are fasting and non-fasting, optionally wherein the non-fasting is post-glucose consumption. In these embodiments, the methods can include comparing the levels in the two states, e.g., comparing the level in a subject in a fasting state to a level in a non-fasting state (e.g., after a glucose challenge such as an OGTT), and optionally calculating a ratio of the levels in the two states. That ratio can then be compared to a reference ratio, e.g., a reference ratio that represents a threshold ratio, above which the subject has an increased risk of developing a glucose-related metabolic disorder, e.g., diabetes.

According to another aspect of the invention, methods for stratifying a population are provided. The methods include (i) determining levels or occurrences of a plurality of biomarkers for a plurality of subjects of a population wherein the plurality of biomarkers are selected from an amino acid, a glucose metabolite, a ketone body, a lipid metabolite, and a bile acid; and (ii) stratifying the plurality of subjects based on the levels or occurrences of the plurality of biomarkers.

In some embodiments, the population comprises subjects selected from: subjects at risk of having a glucose-related metabolic disorder, subjects having a glucose-related metabolic disorder, subjects suspected of having a glucose-related metabolic disorder, and subjects not having a glucose-related metabolic disorder. In certain embodiments, the glucose-related metabolic disorder is diabetes, impaired fasting glycemia, impaired glucose tolerance, polycystic ovary syndrome, or Metabolic Syndrome. In certain embodiments, the diabetes is selected from type I diabetes, type II diabetes, gestational diabetes, and another specific type of diabetes. In certain embodiments, the other specific type of diabetes is associated with a genetic defect, a genetic syndrome, a genetically determined abnormality, an exocrine pancreas defect, an endocrinopathy, a drug or chemical cause, an infection, or an immunological pathogenesis different from that which leads to Type 1 diabetes.

According to another aspect of the invention, methods are provided for selecting a subject for a study. The method include (i) determining levels or occurrences of a plurality of biomarkers in the subject, wherein the plurality of biomarkers are selected from an amino acid, a glucose metabolite, a ketone body, a lipid metabolite, and a bile acid; and (ii) selecting the subject for the study based of the levels or occurrences of the plurality of biomarkers in the subject. In some embodiments, the study is a clinical study. In certain embodiments, the clinical study is to evaluate a treatment for a glucose-related metabolic disorder. In certain embodiments, the treatment is to administer to the subject an effective amount of at least one anti-diabetes compound and/or to instruct the subject to adopt at least one lifestyle change. In certain embodiments, the at least one anti-diabetes compound is selected from an alpha-glucosidase inhibitor, a biguanide, a meglitinide, a sulfonylurea, a thiazolidinedione, an amylin, a glucagon-like peptide I, a vanadate (vanadyl), a dichloroacetic acid, a carnitine palmitoyltransferase inhibitor, a B₃ adrenoceptor agonist, a peptide analog, a DPP-4 inhibitor, dichloroacetic acid and insulin.

According to another aspect of the invention, kits for evaluating biomarkers in a subject are provided. The kits include (i) reagents suitable for determining levels of two or more metabolic biomarkers in a sample, wherein the biomarkers are selected from an amino acid, a glucose metabolite, a ketone body, a lipid metabolite, and a bile acid; (ii) optionally one or more control samples, wherein a comparison between the levels or occurrences of the two or more biomarkers in the subject and levels or occurrences of the two or more biomarkers in the one or more control samples is indicative of a clinical status; and (iii) optionally indicia providing predetermined levels or occurrences, wherein a comparison between the levels or occurrences of the two or more biomarkers in the subject and the predetermined levels or occurrences is indicative of a clinical status.

According to another aspect of the invention, methods for selecting a treatment for a subject having, or suspected of having, a glucose-related metabolic disorder are provided. The methods include determining levels a plurality of biomarkers in a clinical sample obtained from the subject, wherein the plurality of biomarkers are selected from: an amino acid, a glucose metabolite, a ketone body, a lipid metabolite, and a bile acid, and wherein the levels of the plurality of biomarkers are indicative of the suitability of a treatment for the glucose-related metabolic disorder in the subject. In some embodiments, the treatment is to administer to the subject an effective amount of at least one anti-diabetes compound and/or to instruct the subject to adopt at least one lifestyle change. In certain embodiments, the at least one anti-diabetes compound is selected from an alpha-glucosidase inhibitor, a biguanide, a meglitinide, a sulfonylurea, a thiazolidinedione, an amylin, a glucagon-like peptide I, a vanadate (vanadyl), a dichloroacetic acid, a carnitine palmitoyltransferase inhibitor, a B₃ adrenoceptor agonist, a peptide analog, a DPP-4 inhibitor, dichloroacetic acid and insulin.

In some embodiments of the methods described herein, the methods include monitoring levels of the plurality of biomarkers in a subject over time. In some embodiments, a treatment can be adjusted in response to changes in the levels of the plurality of biomarkers in the subject over time.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 depicts the metabolic response to OGTT in MACS (normal glucose tolerance). (A) Glucose and insulin (mean±s.e.m.). (B) Magnitude and significance of metabolite change over time. Dots represent the 97 metabolites detected in plasma. For each time point, median fold change from fasting levels is plotted against the significance of change. Significant (p<0.001) changes are in red. (C) Metabolites that changed significantly in response to glucose ingestion. The temporal patterns of the 21 metabolites that changed significantly (p<0.001) from their fasting levels and showed a significantly (p<0.05) distinct response compared to control (water ingestion) are shown on a color scale. Color intensity reflects the median fold change. Metabolites are ordered according to the magnitude of change. Values were truncated at 8 for color contrast.

FIG. 2 depicts validation of metabolite response at the 2-hour time point. For 18 metabolite responses which replicated significantly (p<0.05) in FOS-NGT, the magnitude of change in FOS-NGT is plotted against the magnitude of change in MACS. Dots correspond to the median fold change at 2-hour. Abbreviations: TCDCA, taurochenodeoxycholic acid; GCDCA, glycochenodeoxycholic acid; GCA, glycocholic acid, Orn: ornithine, Cit: citrulline, β-OH-B: β-hydroxybutyrate.

FIG. 3 depicts metabolic responses not previously linked to glucose homeostasis. Kinetic patterns in MACS are shown (mean±s.e.m.). (A) Bile acids. Abbreviations: TCDCA, Taurochenodeoxycholic acid; GCDCA, Glycochenodeoxycholic acid; GCA, Glycocholic acid. (B) Citrulline and ornithine, urea cycle intermediates. (C) Hypoxanthine, a product of purine nucleotide degradation.

FIG. 4 depicts metabolites reflecting 4 arms of insulin action. (A) Four arms of insulin action and their associated metabolic markers. (B) Stimulation of glucose metabolism. Kinetic patterns in MACS are shown (mean±s.e.m.). (C) Suppression of catabolism. Each line corresponds to metabolite levels of a subject. The arrow marks the median time to half-maximal decrease. The inter-quartile range of metabolite levels is yellow-shaded. 12 MACS subjects profiled in the same LC-MS/MS experiment are shown. Abbreviations: β-OH-B, β-hydroxybutyrate.

FIG. 5 depicts correlation between fasting insulin and 2-hour metabolite changes in FOS-NGT (impaired glucose tolerance). (A) 2-hour changes in markers of insulin action are correlated with fasting insulin concentration. Each dot corresponds to a subject. (B) Regression models predicting fasting insulin. Δ denotes log of the 2-hour fold change. (C) A bivariate model consisting of the 2-hour decline of Leu/Ile and glycerol.

### DETAILED DESCRIPTION

To obtain a systematic view of the physiologic response to glucose ingestion in health and disease, the concentrations of a large and diverse set of metabolites were simultaneously monitored. This integrated analysis facilitates the classification of metabolic states, reveals new pathways, and improves the sensitivity for detection of abnormalities. Traditionally, single metabolites or classes of small molecules have been detected using dedicated analytical assays. With these methods, relationships between diverse metabolites and pathways may be missed, and a comprehensive picture of a complex physiologic program has not been possible. Such relationships are important in understanding disease pathogenesis and in aiding in the diagnosis of disease.

Recent advances in analytical chemistry and computation, such as Nuclear magnetic resonance (NMR) spectroscopy and mass spectrometry (MS), facilitate the measurement of metabolites in biological samples (Dunn et al., Analyst 130(5):606-625 (2005)). While NMR spectroscopy has a number of advantages, primarily its non-destructive nature and its ability to provide information on chemical structure, it tends to have low sensitivity. MS technology, on the other hand, affords sensitive and specific analysis of metabolites in complex biological samples, particularly when implemented as tandem mass spectrometry and coupled with high performance liquid chromatography (LC), a combination termed LC-MS/MS. Metabolic profiling with LC-MS/MS technology has already been successfully used for identifying human plasma markers of myocardial ischemia (Sabatine et al., Circulation 112(25):3868-3875 (2005)) as well as for characterizing the metabolic response to starvation in model organisms (Brauer et al., Proc Natl Acad Sci USA 103(51):19302-19307 (2006)).

described herein is a metabolic profiling system capable of quantifying metabolites in plasma (see, e.g., Example 5), and its application to, among other things, studying the human response to an oral glucose load. In some embodiments, the methods use LC-MS/MS. As described herein, an initial population including 191 endogenous human metabolites spanning diverse chemical classes was measured, including amino acids, nitrogenous compounds and amines (32%); purines and pyrimidines (26%); organic acids (11%); carbohydrates and sugar phosphates (8%); vitamins (7%); bile acids (3%); phosphate acids and phosphate alcohols (2%); and others (11%) (classification was based on the chemical taxonomy annotation in the Human Metabolome Database (Wishart et al., Nucleic Acids Res 35, D521-6 (2007)). This collection of 191 metabolites included those previously studied in the context of glucose homeostasis, as well as many metabolites not previously linked to this area. The technology was first applied to healthy subjects in order to characterize the normal human response to an oral glucose challenge; then to a cohort of subjects with impaired glucose tolerance to evaluate the effects of reduced insulin sensitivity; and then to subjects with diabetes mellitus.

As described herein, metabolic profiling was applied to investigate the kinetics of human plasma biochemicals in response to an oral glucose challenge, and to characterize this physiologic program in a multidimensional way. The systematic approach confirmed known polar metabolite changes associated with the OGTT, while spotlighting some pathways never linked to this program. Importantly, simultaneous measurement of multiple metabolites made it possible to explore connections between metabolic pathways, providing novel insights into normal physiology and disease.

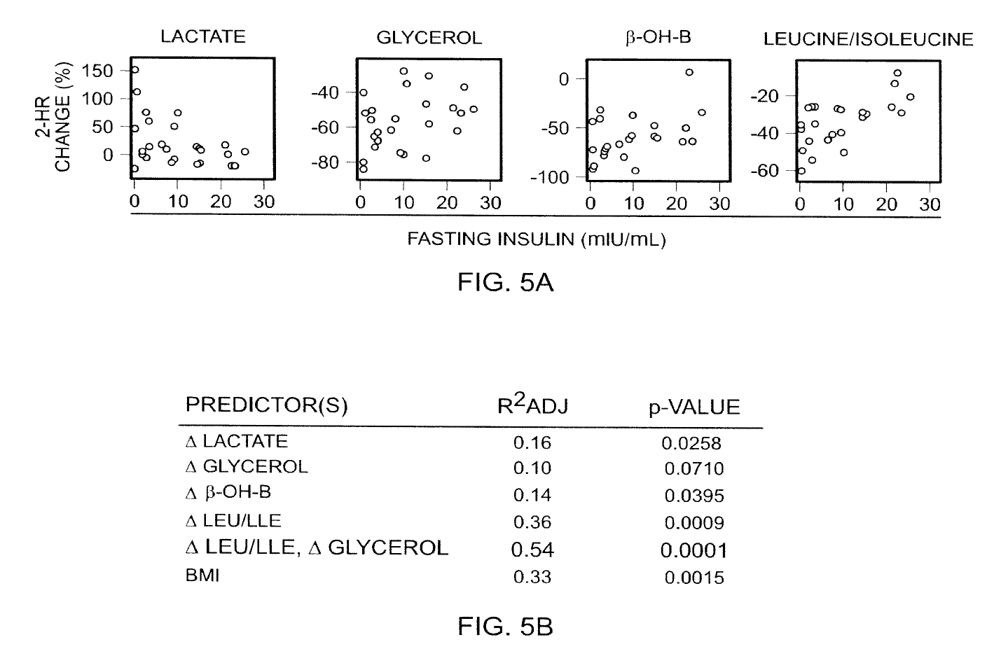

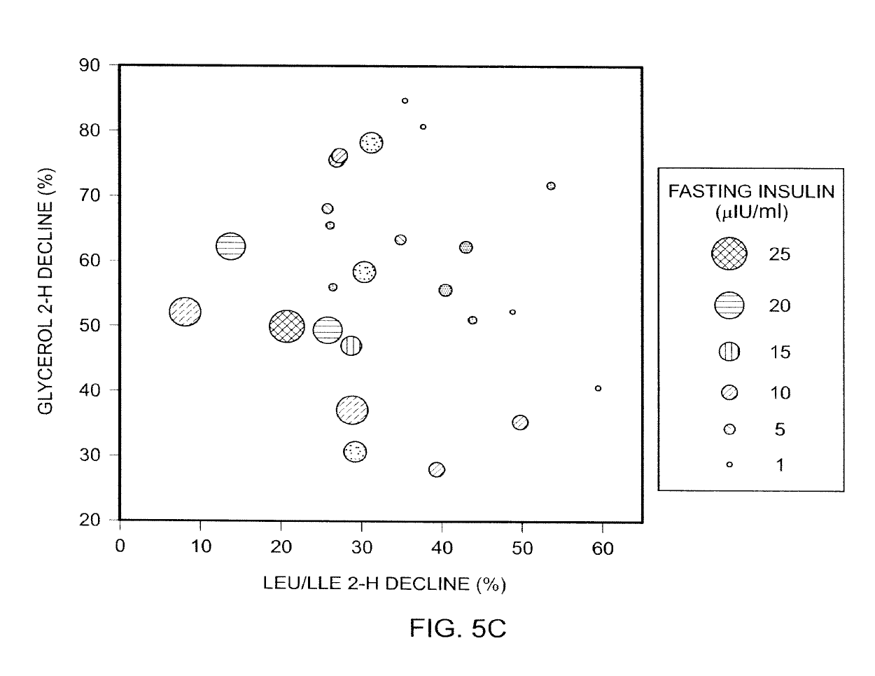

Resistance to the action of insulin, in principle, may develop in multiple physiological axes. In prior studies, for example, inadequate suppression of lipolysis was observed in women with a history of gestational diabetes (Chan et al., Clin Endocrinol (Oxf) 36(4):417-420 (1992)), and an elevated proteolysis rate was seen in subjects with HIV-associated insulin resistance (Reeds et al., Diabetes 55(10):2849-2855 (2006)). In obesity, manifestations of insulin resistance include elevated rates of lipolysis (Robertson et al., Int J Obes 15(10):635-645 (1991)) and proteolysis (Jensen and Haymond, Am J Clin Nutr 53(1):172-176 (1991); Luzi et al., Am J Physiol 270(2 Pt 1):E273-281 (1996)). In the present study, metabolite patterns were identified that reflect a loss of sensitivity in four distinct arms of insulin action (FIG. 5A): dampened increase in lactate (glucose metabolism), diminished reductions of glycerol and β-hydroxybutyrate (suppression of lipolysis and ketogenesis, respectively) and blunted decrease in amino acids (inhibition of proteolysis). The four distinct arms of insulin action can vary independently in different subjects. Remarkably, this wealth of metabolic variation was detected within a cohort bearing a uniform diagnosis of impaired glucose tolerance (FOS-IGT, Table 2). Thus simultaneous measurement of multiple metabolites that cover the four distinct arms of insulin action can be a powerful tool for refining the physiologic, diagnostic, and clinical picture of insulin resistance.

The profiling approaches of the present invention have not only revealed multiple manifestations of insulin resistance, but have also allowed the exploration of their interplay. Changes in Leu/Ile levels and glycerol levels jointly predicted fasting insulin levels, a indicator of insulin sensitivity, and each metabolite offered complementary and significant predictive power (FIG. 5C). This complementation supports the notion of selective insulin resistance: some subjects exhibit more resistance in proteolysis, while others are more resistant in lipolysis. Recently, Brown and Goldstein described a pathogenic role for selective insulin resistance, where in diabetic mice insulin failed to suppress gluconeogensis, but at the same time continued to activate lipogenesis (Brown and Goldstein, Cell Metab 7(2):95-96 (2008)). Our findings suggest that monitoring multiple arms of insulin action by measuring metabolite markers during, for example, a clinical OGTT could facilitate the classification of subjects on multiple axes of insulin action. Such measurements could assist in the early diagnosis of type-II diabetes mellitus (T2DM), and could also be useful in monitoring therapeutic response and thereby guiding treatment.

Glucose-Related Metabolic Disorders

The invention, in some aspects, relates to methods, compositions and kits useful for diagnosing and determining risk of developing glucose-related metabolic disorders. As used herein, “glucose-related metabolic disorders” refer broadly to any disorder, disease, or syndrome characterized by a deficiency in the regulation of glucose homeostasis (e.g., hyperglycemia). Typically a glucose-related metabolic disorder is associated with abnormal insulin levels, insulin activity, and/or sensitivity to insulin (e.g., insulin resistance). As used herein diabetes (also referred to as diabetes mellitus), refers to any one of a number of exemplary classes (or types) of glucose-related metabolic disorders. Diabetes includes, but is not limited to the following classes (or types): type I diabetes mellitus, type II diabetes mellitus, gestational diabetes, and other specific types of diabetes. Glucose-related metabolic disorders also include prediabetic conditions, such as those associated with impaired fasting glycemia and impaired glucose tolerance. Glucose-related metabolic disorders are often associated with symptoms in a subject such as increased thirst and urine volume, recurrent infections, unexplained weight loss and, in severe cases, drowsiness and coma; high levels of glycosuria are often present. Children suspected of having a glucose-related metabolic disorder may, in some cases, present with severe symptoms, such as high blood glucose levels, glycosuria, and/or ketonuria.

Type 1 diabetes is usually due to autoimmune destruction of the pancreatic beta cells. Type 2 diabetes is characterized by insulin resistance in target tissues, which may result in a need for abnormally high amounts of insulin and diabetes develops when the beta cells cannot meet this demand. Gestational diabetes is similar to type 2 diabetes in that it involves insulin resistance; the hormones of pregnancy can cause insulin resistance in women genetically predisposed to developing this condition. Other specific types of diabetes are known in the art and disclosed in Definition, Diagnosis and Classification of Diabetes Mellitus and its Complications, Report: WHO/NCD/NCS/99.2 by the World Health Organisation, Department of Noncommunicable Disease Surveillance (1999), the contents of which are incorporated herein in their entirety by reference.

In some embodiments, the glucose-related metabolic disorder is type 1 diabetes. Type 1 diabetes is also referred as the autoimmune diabetes mellitus form of diabetes, insulin-dependent diabetes, or juvenile-onset diabetes, and is associated with the processes of beta-cell destruction that may ultimately lead to a state in which insulin is required to prevent the development of ketoacidosis, coma and death. In some embodiments, the glucose-related metabolic disorder is Type 2 diabetes. Type 2 is also referred to as non-insulin-dependent diabetes or adult-onset diabetes, and is characterized by disorders of insulin action and insulin secretion, either of which may be the predominant feature. Both are usually present at the time that this form of diabetes is clinically manifest.

In some embodiments, the glucose-related metabolic disorder is gestational hyperglycemia or gestational diabetes. These are forms of diabetes associated with pregnancy. Gestational diabetes is associated with carbohydrate intolerance resulting in hyperglycemia of variable severity with onset or first recognition during pregnancy. Thus, it does not exclude the possibility that the glucose intolerance may antedate the pregnancy but was previously unrecognized. The classification typically applies irrespective of whether or not insulin is used for treatment or the condition persists after pregnancy. In some embodiments, a glucose-related metabolic disorder is “Metabolic Syndrome” which is often characterized by hypertension, central (upper body) obesity, and dyslipidaemia, with or without hyperglycaemia. Subjects with the Metabolic Syndrome are at high risk of macrovascular disease. Often a person with abnormal glucose tolerance will be found to have at least one or more of the other cardiovascular disease (CVD) risk components. The Metabolic Syndrome is also referred to as Syndrome X and the Insulin Resistance Syndrome. Epidemiological studies confirm that this syndrome occurs commonly in a wide variety of ethnic groups including Caucasians, African-Americans, Mexican-Americans, Asian Indians, Chinese, Australian Aborigines, Polynesians and Micronesians. The Metabolic Syndrome with normal glucose tolerance identifies a subject as a member of a group at very high risk of diabetes. Thus, vigorous early management of the syndrome may have a significant impact on the prevention of both diabetes- and cardiovascular disease.

Diagnosis/Characterization

The present invention relates to methods useful for the characterization (e.g., clinical evaluation, diagnosis, classification, prediction, profiling) of glucose-related metabolic disorders, such as diabetes, based on the levels or occurrence of certain metabolites referred to herein as biomarkers, or metabolic biomarkers. As used herein, levels refer to the amount or concentration of a metabolite in a sample (e.g., a plasma sample) or subject. Whereas, occurrence refers to the presence or absence of a detectable metabolite in a sample. Thus, level is a continuous indicator of amount, whereas occurrence is a binary indicator of a metabolite. In some cases, an occurrence may be determined using a threshold level above which a biomarker is present and below which a biomarker is absent.

The metabolic biomarkers described herein are particularly useful for characterizing a glucose-related metabolic disorder. In some embodiments, the biomarkers may be amino acids, glucose metabolites, ketone bodies, lipid metabolites, or bile acids. In some cases, the metabolic biomarkers reflect insulin's action on proteolysis, lipolysis, ketogenesis, or glycolysis. Useful biomarkers also include urea cycle intermediates (e.g., citrulline or ornithine) and purine degradation products (e.g., hypoxanthine and xanthine).

The invention relates to the discovery of a plurality of biomarkers that are useful for characterizing a glucose-related metabolic disorder. The number of biomarkers, or metabolites, in the plurality (at least two) may be 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more, e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more. It will often be useful for the plurality of metabolic biomarkers to comprise metabolites from each of four axes of insulin action: proteolysis, lipolysis, ketogenesis, and glycolysis. For example, a metabolite from the glycolysis axis can be, e.g., a glucose metabolite such as glucose, pyruvate, lactate, and/or malate. A metabolite from the ketogenesis axis can be, e.g., beta-hydroxybutyrate, acetoacetate, and/or acetone. A metabolite from the proteolysis axis can be, e.g., an amino acid such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and/or valine. In some cases, a metabolite from the proteolysis axis is a branched chain amino acid. A metabolite from the lipolysis axis may be glycerol. The metabolite may also be a non-proteinogenic amino acid, such as citrulline or ornithine.

In specific embodiments, the biomarkers are selected from: In some embodiments, the metabolic biomarkers are selected from: (R)-3-Hydroxybutanoate [b-hydroxybutyrate]; (S)-Lactate [Lactate]; (S)-Malate [Malate]; Glucose; Glycerol; Glycochenodeoxycholate [GCDCA]; Glycocholate [GCA]; Hippurate [Hippuric acid]; Hypoxanthine; L-Arginine [Arginine]; L-Citrulline [Citrulline]; Leucine/Isoleucine; L-Histidine [Histidine]; L Lysine [Lysine]; L-Methionine [Methionine]; L-Ornithine [Ornithine]; L-Phenylalanine [Phenylalanine]; L Tyrosine [Tyrosine]; Pyruvate; Taurochenodeoxycholate [TCDCA]; and Valine.

In some embodiments, the metabolic biomarkers are selected from Hippuric acid, Taurochenodeoxycholic acid (TCDCA), Glycochenodeoxycholic acid (GCDCA), Glycocholic acid (GCA), Lactate, Glucose, Pyruvate, Malate, Valine, Histidine, Lysine, Phenylalanine, Arginine, Ornithine, Omithine, Tyrosine, Leucine, Isoleucine, Methionine, Citrulline, Hypoxanthine, Glycerol, and Beta-Hydroxybutyrate.

In some embodiments, the metabolic biomarkers are selected from β-hydroxybutyrate, Citrulline, Glycerol, Glycochenodeoxycholic acid, Glycocholic acid, Hippuric acid, Histidine, Hypoxanthine, Lactate, Leucine/Isoleucine, Lysine, Methionine, Ornithine, Phenylalanine, Pyruvate, Taurochenodeoxycholic acid, Tyrosine, and Valine.

In some embodiments, the methods involve determining the occurrences or levels of a plurality of metabolic biomarkers in a clinical sample, comparing the result to a reference, and characterizing (e.g., diagnosing, classifying) the sample based on the results of the comparison. A clinical sample can be any biological specimen (e.g., a blood sample) useful for characterizing the glucose-related metabolic disorder (e.g., diabetes). Typically, a clinical sample contains one or more metabolites. Exemplary biological specimens can include blood, serum, plasma, or urine. In preferred embodiments, a clinical sample is a blood (plasma) or urine sample.

In some embodiments, clinical samples are obtained from subjects (also referred to herein as individuals). As used herein, a subject is a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. In some embodiments, a subject is a diabetic animal model. Diabetes animal models are well known in the art, for example: Leiter, Curr Protoc Immunol. 2001 May; Chapter 15:Unit 15.9; Levine et al., Am J Physiol Regul Integr Comp Physiol. 2008 Apr. 16; Oh YS, et al., Diabetologia. 2008 Apr. 12; Sasaki et al., Arterioscler Thromb Vasc Biol. 2008 Apr. 10; Beauquis et al., Exp Neurol. 2008 April; 210(2):359-67; Cheng et al., Mol Pharm. 2008 January-February; 5(1):77-91; Tikellis et al., Atherosclerosis. 2007 Dec. 17; Novelli et al., Pancreas. 2007 November; 35(4):e10-7; and Khazaei et al., Physiol Res. 2007 Nov. 30. Preferred subjects are humans (huma subjects). The huma subject may be a pediatric or adult subject. In some embodiments the adult subject is a geriatric subject.

In some embodiments, the methods involve diagnosing glucose-related metabolic disorder in a subject. To practice the diagnostic methods the levels of a plurality of biomarkers are typically determined. These levels are compared to a reference wherein the levels of the plurality of biomarkers in comparison to the reference is indicative of whether or not the subject has a glucose related metabolic disorder and/or should be diagnosed with the glucose related metabolic disorder.

As used herein, diagnosing includes both diagnosing and aiding in diagnosing. Thus, other diagnostic criteria may be evaluated in conjunction with the results of the methods herein in order to make a diagnosis.

The methods described herein are also useful for assessing the likelihood (or risk) of, or aiding in assessing the likelihood (or risk) of, a subject having or developing a glucose-related metabolic disorder. To practice the methods levels of a plurality of biomarkers are typically determined. These levels are compared to a reference wherein the levels of the plurality of biomarkers in comparison to the reference is indicative of the likelihood that the subject will develop a glucose related metabolic disorder.

Other criteria for assessing likelihood that are known in the art (e.g., Body Mass Index (BMI), family history) can also be evaluated in conjunction with the methods described herein in order to make a complete likelihood assessment.

In some embodiments, methods involve determining the glucose control capacity or insulin sensitivity of a subject. To practice the methods, typically the levels of a plurality of biomarkers are determined. These levels are compared to a reference wherein the levels of the plurality of biomarkers in comparison to the reference are indicative of the glucose control capacity or insulin sensitivity.

As used herein, insulin sensitivity refers to the responsiveness of a subject, or cells of a subject, to the effects of insulin. For example, subjects with insulin resistance are less sensitive to insulin and therefore, have low insulin sensitivity. Techniques for measuring insulin sensitivity are well known in the art and include, for example, the hyperinsulinemic euglycemic clamp (i.e., the “clamp” technique), the Modified Insulin Suppression Test, fasting insulin levels, and glucose tolerance tests (e.g., an Oral Glucose Tolerance Test). The methods disclosed herein are also useful to characterize and obtain further insight on insulin sensitivity.

As used herein, glucose control capacity refers to a subject's ability (capacity) to control glucose levels within homeostatic limits (a physiologically safe/normal range). Consequently, insulin (and therefore insulin sensitivity), among other things, influences a subject's glucose control capacity. Other regulatory factors (e.g., hormones) in addition to insulin, such as glucagon, that influence glucose control capacity of a subject are well known in art. The methods disclosed herein are useful to characterize and obtain further insight on glucose control capacity.

The levels of the metabolites for a subject can be obtained by any art recognized method. Typically, the level is determined by measuring the level of the metabolite in a body fluid (clinical sample), e.g., blood, plasma, or urine. The level can be determined by any method known in the art, e.g., ELISA, immunoassays, enzymatic assays, spectrophotometry, colorimetry, fluorometry, bacterial assays, liquid chromatography, gas chromatography, mass spectrometry, Liquid chromatography-mass spectrometry (LC-MS), LC-MS/MS, tandem MS); high pressure liquid chromatography (HPLC), HPLC-MS, and nuclear magnetic resonance spectroscopy or other known techniques for determining the presence and/or quantity of a metabolite. Conventional methods include sending a clinical sample(s) to a commercial laboratory for measurement or the use of commercially available assay kits. Commercially available assay kits are known in the art. For example, Quest Diagnostics, Sigma Aldrich, CATACHEM Inc., Eton Bioscience Inc., and BioVision Research Products are exemplary suppliers of such assays. Specific examples of commercially available assay kits include lactate assay kits (e.g., Quest Diagnostics code: 25247W), β-hydroxybutyrate assay kits (e.g., Quest Diagnostics code: 37054N), free glycerol determination kit (e.g., Sigma Aldrich code: FG0100), leucine/isoleucine assay kits (e.g., Quest Diagnostics code: 767×), and bile acid (GCA, GCDCA, TCDCA) assay kits (e.g., Quest Diagnostics code: 8482N). Other exemplary kits and suppliers will be apparent to the skilled artisan.

In some cases, the methods disclosed herein involve comparing levels or occurrences to a reference. The reference can take on a variety of forms. In some cases, the reference comprises predetermined values for a plurality of metabolites (e.g., each of the plurality of metabolites). The predetermined value can take a variety of forms. It can be a level or occurrence of a metabolite in a control subject (e.g., a subject with a glucose-related metabolic disorder (i.e., an affected subject) or a subject without such a disorder (i.e., a normal subject)). It can be a level or occurrence of a metabolite in a fasting subject. It can be a level or occurrence in the same subject, e.g., at a different time point. A predetermined value that represent a level(s) of a metabolite is referred to herein as a predetermined level. A predetermined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

Subjects associated with predetermined values are typically referred to as control subjects (or controls). A control subject may or may not have a glucose related metabolic disorder (e.g., diabetes). In some cases it may be desirable that control subject is a diabetic, and in other cases it may be desirable that a control subject is a non-diabetic. Thus, in some cases the level of a metabolite in a subject being greater than or equal to the level of the metabolite in a control subject is indicative of a clinical status (e.g., indicative of a glucose-related metabolic disorder diagnosis). In other cases the level of a metabolite in a subject being less than or equal to the level of the metabolite in a control subject is indicative of a clinical status. The amount of the greater than and the amount of the less than is usually of a sufficient magnitude to, for example, facilitate distinguishing a subject from a control subject using the disclosed methods. Typically, the greater than, or the less than, that is sufficient to distinguish a subject from a control subject is a statistically significant greater than, or a statistically significant less than. In cases where the level of a metabolite in a subject being equal to the level of the metabolite in a control subject is indicative of a clinical status, the “being equal” refers to being approximately equal (e.g., not statistically different).

The predetermined value can depend upon a particular population of subjects (e.g., huma subjects) selected. For example, an apparently healthy population will have a different ‘normal’ range of metabolites than will a population of subjects which have, or are likely to have, a glucose-related metabolic disorder. Accordingly, the predetermined values selected may take into account the category (e.g., healthy, at risk, diseased) in which a subject (e.g., huma subject) falls. Appropriate ranges and categories can be selected with no more than routine experimentation by those of ordinary skill in the art.

In some cases a predetermined value of a metabolic biomarker is a value that is the average for a population of healthy subjects (huma subjects) (e.g., huma subjects who have no apparent signs and symptoms of a glucose-related metabolic disorder). The predetermined value will depend, of course, on the particular metabolite (biomarker) selected and even upon the characteristics of the population in which the subject lies. In characterizing likelihood, or risk, numerous predetermined values can be established.

A level, in some embodiments, may itself be a relative level that reflects a comparison of levels between two states. For example, a level may be a relative level that reflects a comparison between fasting (e.g., pre-glucose consumption) and non-fasting states (e.g., post-glucose consumption). Where levels are relative levels that reflect a comparison between fasting and non-fasting states, the non-fasting state may be, for example, about 30 minutes, about 60 minutes, about 90 minutes, about 120 minutes, or more, post glucose consumption. In some cases, relative levels may be determined (e.g., by clinical personnel) during a standard oral glucose tolerance test, e.g., a first or baseline level that is obtained before the test and a second level that is obtained after the glucose consumption). Relative levels that reflect a comparison (e.g., ratio, difference, logarithmic difference, percentage change, etc.) between two states (e.g., fasting and non-fasting) may be referred to as delta values. For example, in the case of an oral glucose tolerance test, delta values may be a percentage change in levels of a biomarker from fasting to non-fasting states. The use of relative levels is beneficial in some cases because, to an extent, they exclude measurement related variations (e.g., laboratory personnel, laboratories, measurements devices, reagent lots/preparations, assay kits, etc.). However, the invention is not so limited.

In some aspects of the invention, delta values of metabolites between a fasting state and a 2 hour post-glucose ingestion state have been predetermined. These predetermined values are shown in Table 1 below, as a percent change from the fasting level, and are presented as mean±standard deviation (range). Predetermined values are listed for three human cohorts: MACS (normal, n=22), FOS-NGT (normal, n=25) and FOS-IGT (impaired glucose tolerance, n=25). Description of the cohorts is provide in Table 2.

TABLE 1
Predetermined Values Of Metabolites Between a Fasting State and a
2 Hour Post-Glucose Ingestion State (Percent Change)

| | | | FOS-IGT |
|---|---|---|---|
| | | | (impaired |
| | MACS | FOS-NGT | glucose |
| | (normal, | (normal, | tolerance, |
| Metabolite | n = 22) | n = 25) | n = 25) |
| β-hydroxybutyrate | −55 ± 25 | −44 ± 29 | −59 ± 22 |
| | (−86, −11) | (−85, 67) | (−93, 7) |
| Citrulline | −34 ± 12 | −37 ± 13 | −46 ± 10 |
| | (−56, −5) | (−60, −8) | (−64, −28) |
| Glycerol | −52 ± 32 | −53 ± 17 | −57 ± 16 |
| | (−86, 59) | (−76, −13) | (−85, −28) |
| Glycochenodeoxycholic acid | 225 ± 221 | 286 ± 388 | 113 ± 207 |
| | (−73, 787) | (−65, 1570) | (−89, 887) |
| Glycocholic acid | 174 ± 183 | 208 ± 268 | 114 ± 274 |
| | (−76, 631) | (−65, 716) | (−83, 954) |
| Hippuric acid | 686 ± 514 | 526 ± 488 | 370 ± 315 |
| | (249, 2062) | (22, 2033) | (16, 1128) |
| Histidine | −14 ± 15 | −9 ± 11 | −18 ± 17 |
| | (−42, 17) | (−28, 16) | (−39, 11) |
| Hypoxanthine | −37 ± 23 | −14 ± 90 | −25 ± 50 |
| | (−81, 15) | (−82, 278) | (−90, 60) |
| Lactate | 40 ± 39 | 34 ± 40 | 22 ± 44 |
| | (−39, 123) | (−18, 142) | (−24, 152) |
| Leucine/Isoleucine | −33 ± 10 | −31 ± 9 | −33 ± 12 |
| | (−56, −16) | (−48, −9) | (−59, −8) |
| Lysine | −18 ± 10 | −10 ± 10 | −18 ± 8 |
| | (−41, −2) | (−28, 13) | (−31, −0) |
| Methionine | −33 ± 10 | −28 ± 10 | −32 ± 11 |
| | (−48, −15) | (−46, −1) | (−54, −3) |
| Ornithine | −27 ± 11 | −24 ± 12 | −29 ± 10 |
| | (−44, −4) | (−44, −4) | (−41, −5) |
| Phenylalanine | −20 ± 9 | −24 ± 8 | −26 ± 8 |
| | (−37, 1) | (−43, −10) | (−41, −10) |
| Pyruvate | 22 ± 24 | 25 ± 49 | 19 ± 42 |
| | (−27, 67) | (−29, 223) | (−49, 106) |
| Taurochenodeoxycholic acid | 266 ± 305 | 363 ± 487 | 121 ± 247 |
| | (−80, 1009) | (−70, 1716) | (−83, 960) |
| Tyrosine | −31 ± 12 | −31 ± 11 | −30 ± 16 |
| | (−52, −6) | (−50, −5) | (−52, 4) |
| Valine | −15 ± 9 | −14 ± 6 | −16 ± 9 |
| | (−42, −1) | (−23, −2) | (−33, 6) |

The values in Table 1 represent percent change from the fasting levels.

### Metabolic Profiling

The invention, in some aspects, relates to methods useful for metabolic profiling of subjects who have or are suspected or at risk of having a glucose-related metabolic disorder. In some aspects, the invention relates to characterizing glucose-related metabolic disorders using metabolic profiles. In some embodiments, the invention relates to diagnosing and characterizing diabetes (e.g., Type II diabetes) using metabolic profiles.

Glucose-related metabolic disorders (e.g., diabetes) include disorders arising from distinct etiologies for which several classes or types exist (e.g., Type I, Type II, Gestational, and Other Specific Types). As disclosed herein, glucose-related metabolic disorders can be further partitioned into various sub-classes, which may benefit from different treatments. The methods disclosed herein are useful for the identification of disease types, and/or sub-types, and the identification of specific therapies to target each particular disease type, and/or sub-type.

In some aspects, the invention is useful for identifying sub-classes (or sub-types) of glucose-related metabolic disorders based on metabolic profiles. For example, the invention provides methods for assigning a clinical sample (e.g., a serum sample) to a known etiological diabetes class, or sub-class, by evaluating the occurrence or level of a metabolites in the sample (i.e., by metabolic profiling). In some embodiments, the methods can be used for the classification of glucose-related metabolic disorders based on the simultaneous monitoring of a plurality of metabolites, e.g., using LC-MS/MS technology. As one of skill in the art will appreciate, other technologies or approaches known in the art to be suitable for monitoring the levels or occurrences of multiple metabolites in parallel can also be used.

As used herein a metabolic profile refers to a set of occurrences or levels of a plurality (e.g., two or more, four or more) metabolites (biomarkers) which may be used to classify (or sub-classify) a sample, preferably a clinical sample. In some embodiments, control samples, for which a classification (e.g., Type II diabetes) has already been ascertained, are used to produce known metabolic profiles. In some embodiments, the similarity of a test metabolic profile and a known metabolic profile, is assessed by comparing the occurrence or level of the same metabolite in the test and known metabolic profiles (i.e., metabolite pair). In some cases, a test metabolic profile is compared with one or more members of a plurality of known metabolic profiles, and a known metabolic profile that most closely resembles (i.e. is most similar to) the test metabolic profile is identified. In certain cases, the classification of a known metabolic profile (e.g., Type II diabetes) that is identified as similar to a test metabolic profile is assigned to the test metabolic profile, thereby classifying the clinical sample associated with the test metabolic profile.

In some embodiments the invention relates to classifying a sample (e.g., a clinical sample) obtained from a subject (e.g., a clinical patient) based on a metabolic profile, which comprises the occurrences or levels of a plurality of metabolites in the sample. In particular, the methods involve characterizing a clinical sample (e.g., a blood sample) for the evaluation of a glucose-related metabolic disorder (e.g., Type II diabetes). Sample classification can be performed for many reasons. For example, it may be desirable to classify a sample from a subject to determine whether the subject has a glucose-related metabolic disorder of a particular type or sub-type so that the subject can obtain appropriate treatment. Other reasons for classifying a sample include predicting treatment response (e.g., response to a particular drug or therapy regimen) and predicting phenotype (e.g., the likelihood of developing diabetes). Thus, the applications of the invention are numerous and are not limited to the specific examples described herein. The invention can be used in a variety of applications to characterize (e.g., classify) clinical samples based on the occurrence or level of metabolites in a sample.

In some embodiments, the methods are useful for classifying samples across a range of disease phenotypes based on metabolic profiles. For example, a classification model (e.g., discriminant function, naïve bayes, support vector machine, logistic regression, and others known in the art) may be built based on the metabolite levels or occurrences from various samples from subjects known to have different glucose-related metabolic disorders (e.g., Type I, Type II, Gestational, and other specific types of diabetes) and/or from subjects that do not have a glucose-related metabolic disorder, and used to classify subsequently obtained samples (e.g., clinical samples). In one embodiment, this model is created from a set of two or more metabolites whose levels or occurrences are associated with a particular glucose-related metabolic disorder class distinction (e.g., Type II Diabetes) to be predicted (e.g., diagnosed).

Kits

The invention also provides kits for evaluating biomarkers in a subject. The kits of the invention can take on a variety of forms. Typically, the kits will include reagents suitable for determining levels of a plurality of biomarkers (e.g., those disclosed herein, for example as outlined in Table 2) in a sample. In some cases the plurality of biomarkers are selected from an amino acid, a glucose metabolite, a ketone body, a lipid metabolite, and a bile acid. Optionally, the kits may contain, one or more control samples. Typically, a comparison between the levels of the biomarkers in the subject and levels of the biomarkers in the control samples is indicative of a clinical status (e.g., diagnosis, likelihood assessment, insulin sensitivity, glucose control capacity, etc.). Also, the kits, in some cases, will include written information (indicia) providing a reference (e.g., predetermined values), wherein a comparison between the levels of the biomarkers in the subject and the reference (pre-determined values) is indicative of a clinical status. In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

Treatment

The present methods can also be used for selecting a treatment and/or determining a treatment plan for a subject, based on the occurrence or levels of certain metabolites relevant to the glucose related metabolic disorders. In some embodiments, using the method disclosed herein, a health care provider (e.g., a physician) identifies a subject as having or at risk of having a glucose-related metabolic disorder (e.g., Type II Diabetes) and, based on this identification the health care provider determines an adequate treatment plan for the subject. In some embodiments, using the method disclosed herein, a health care provider (e.g., a physician) diagnoses a subject as having a glucose-related metabolic disorder (e.g., Type II Diabetes) based on the occurrence or levels of certain metabolites in a clinical sample obtained from the subject, and/or based on a classification of a clinical sample obtained from the subject. By way of this diagnosis the health care provider determines an adequate treatment or treatment plan for the subject. In some embodiments, the methods further include administering the treatment to the subject.

In some embodiments, the invention relates to identifying subjects who are likely to have successful treatment with a particular drug dose, formulation and/or administration modality. Other embodiments include evaluating the efficacy of a drug using the metabolic profiling methods of the present invention. In some embodiments, the metabolic profiling methods are useful for identifying subjects who are likely to have successful treatment with a particular drug or therapeutic regiment. For example, during a study (e.g., a clinical study) of a drug or treatment, subjects who have a glucose-related metabolic disorder may respond well to the drug or treatment, and others may not. Disparity in treatment efficacy is associated with numerous variables, for example genetic variations among the subjects. In some embodiments, subjects in a population are stratified based on the metabolic profiling methods disclosed herein. In some embodiments, resulting strata are further evaluated based on various epidemiological, and or clinical factors (e.g., response to a specific treatment). In some embodiments, stratum, identified based on a metabolic profile, reflect a subpopulation of subjects that response predictably (e.g., have a predetermined response) to certain treatments. In further embodiments, samples are obtained from subjects who have been subjected to the drug being tested and who have a predetermined response to the treatment. In some cases, a reference can be established from all or a portion of the metabolites from these samples, for example, to provide a reference metabolic profile. A sample to be tested can then be evaluated (e.g., using a prediction model) against the reference and classified on the basis of whether treatment would be successful or unsuccessful. A company and/or person testing a treatment (e.g., compound, drug, life-style change) could discern more accurate information regarding the types or subtypes of glucose-related metabolic disorders for which a treatment is most useful. This information also aids a healthcare provider in determining the best treatment plan for a subject.

In some embodiments, treatment for the glucose-related metabolic disorder is to administer to the subject an effective amount of at least one anti-diabetes compound and/or to instruct the subject to adopt at least one anti-diabetic lifestyle change. Anti-diabetes compound are well known in the art and some are disclosed herein. Non-limiting examples include alpha-glucosidase inhibitors for example acarbose and miglitol; biguanides for example metformin, phenformin, and buformin; meglitinides for example, repaglinide and nateglinide; sulfonylureas, for example tolbutamide, chlorpropamide, tolazamide, acetohexamide, glyburide, glipizide, glimepiride, and gliclazide; thiazolidinediones, for example troglitazone, rosiglitazone, and pioglitazone; peptide analogs, for example glucagon-like peptide I (GLP1) and analogs thereof (e.g., Exentide, Extendin-4, Liraglutide, gastric inhibitory peptide (GIP) and analogs thereof; vanadates (e.g., vanadyl sulfate); GLP agonists; DPP-4 inhibitors, for example vildagliptin and sitagliptin; dichloroacetic acid; amylin; carnitine palmitoyltransferase inhibitors; B3 adrenoceptor agonists; and insulin. Appropriate anti-diabetic lifestyle changes are also well known in the art. Non-limiting examples include increased physical activity, caloric intake restriction, nutritional meal planning, and weight reduction. However, the invention is not so limited and other appropriate treatments will be apparent to one of ordinary skill in the art.

When a therapeutic agent (e.g., anti-diabetic compound) or other treatment is administered, it is administered in an amount effective to treat an existing glucose-related metabolic disorder or reduce the likelihood (or risk) of a future glucose-related metabolic disorder. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain metabolites (e.g., Isoleucine, Leucine or Glycerol) that are indicative of a glucose-related metabolic disorder. It should be understood that the therapeutic agents of the invention are used to treat and/or prevent glucose-related metabolic disorders. Thus, in some cases, they may be used prophylactically in huma subjects at risk of developing a glucose-related metabolic disorder. Thus, in some cases, an effective amount is that amount which can lower the risk of, slow or perhaps prevent altogether the development of a glucose-related metabolic disorder. It will be recognized when the therapeutic agent is used in acute circumstances, it is used to prevent one or more medically undesirable results that typically flow from such adverse events.

Methods for selecting a suitable treatment and an appropriate dose thereof will be apparent to one of ordinary skill in the art.

### EXAMPLES

The invention is further described in the following examples, which do not limit the scope of the invention described in the claims.

### Example 1

### 18 Plasma Metabolites Change Significantly and Reproducibly During an Oral Glucose Challenge

To systematically characterize the normal biochemical response to glucose ingestion in humans, plasma samples for metabolic profiling were obtained from an ongoing study, Metabolic Abnormalities in College Students (MACS).

MACS subjects were young adults in the age range 18-30 who volunteered for the study during the academic year 2006-7. The subjects underwent a series of metabolic evaluations, including a questionnaire for metabolic syndrome risk factors, indirect calorimetry, measurement of body composition and a fasting blood lipid profile. As part of the metabolic assessment, MACS subjects also underwent a 2-hour oral glucose tolerance test (OGTT) with multiple blood draws. OGTTs were performed as follows. Subjects were admitted for observation after a 10 hour overnight fast. An intravenous catheter was inserted into an antecubital vein or a wrist vein and fasting samples were drawn. Next, each subject ingested a glucose solution (Trutol, 75 g in 296 ml; NERL Diagnostics, East Providence, R.I.) or an identical volume of bottled spring water (Poland Spring Water, Wilkes Barre, Pa.) over a 5 minute period. Additional blood samples were drawn from the inserted catheter 30, 60, 90 and 120 minutes after ingestion. Subjects remained at rest throughout the test.

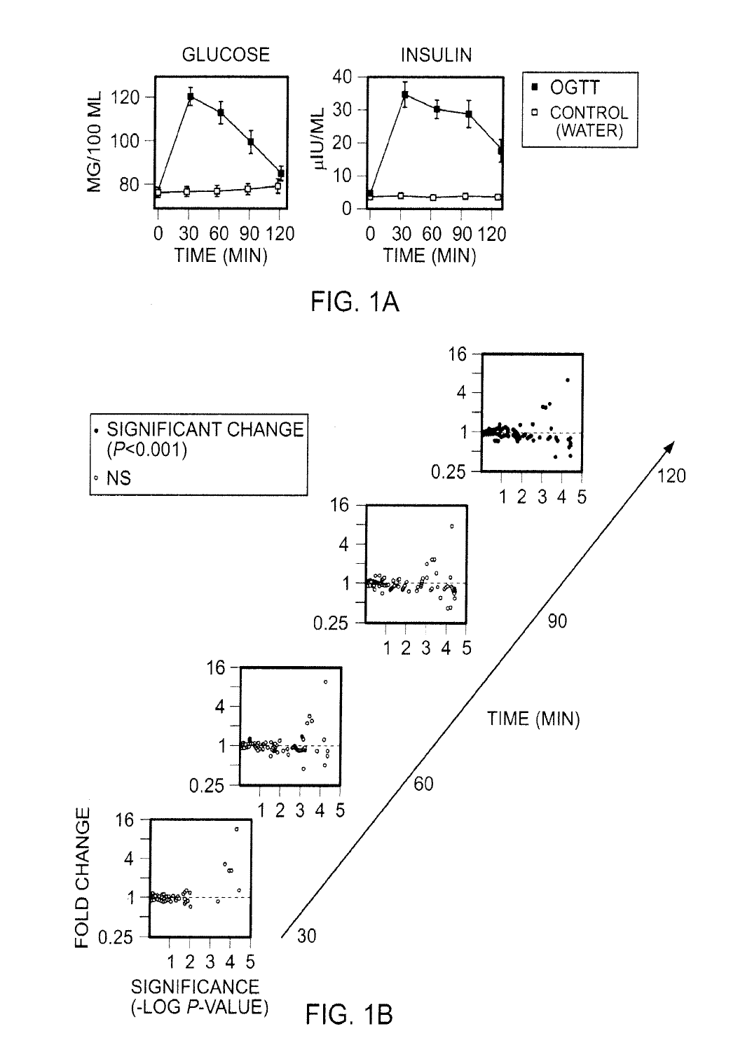

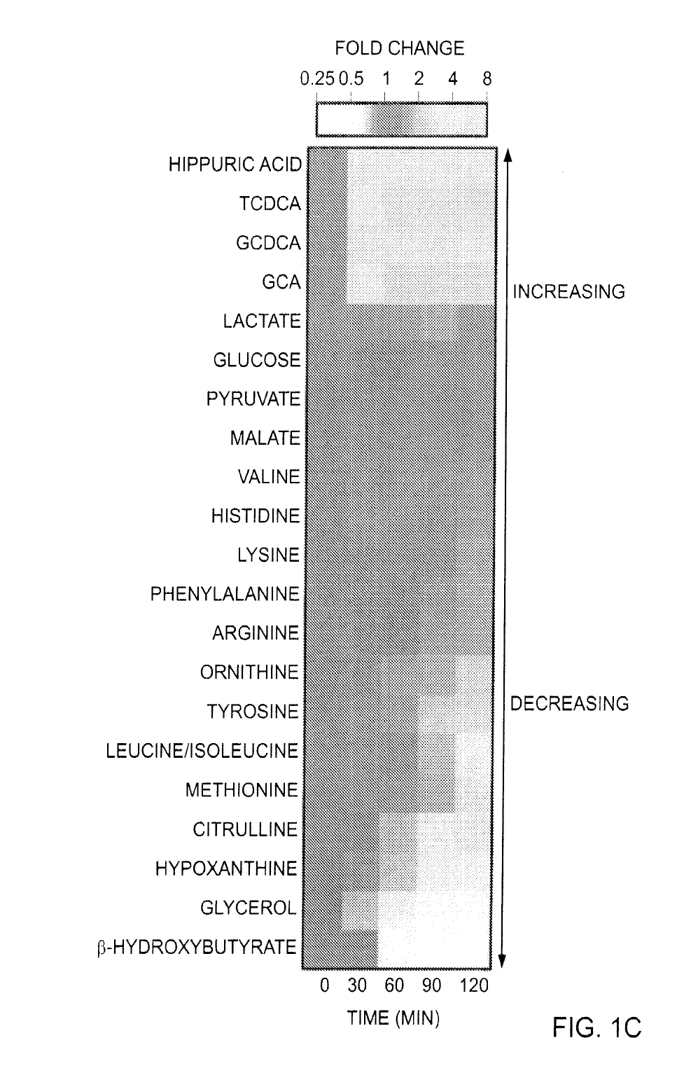

Metabolic profiling analysis was limited to those subjects with normal fasting glucose concentrations (below 100 mg/dL) and normal glucose tolerance (2-hour glucose concentration below 140 mg/dL). To control for the fasting condition and for the effects of fluid ingestion, a subset of MACS subjects selected at random, balanced for gender, were given an identical volume of spring water instead of the glucose solution. Venous blood was drawn during fasting and then every 30 minutes following glucose or water ingestion for the 2-hour duration of the test. Samples were obtained from 22 subjects ingesting glucose and 7 control subjects ingesting water (Table 2). Serum concentrations of glucose and insulin were measured throughout the test (FIG. 1A). All subjects had normal fasting glucose levels, and all glucose-ingesting subjects showed normal glucose tolerance, as currently defined by the American Diabetes Association (American Diabetes Association, 2007).

To validate these findings, fasting and 2-hour OGTT samples were profiled from an independent cohort. The 2-hour time point has clinical significance, since the diagnosis of impaired glucose tolerance and diabetes is dependent on it (American Diabetes Association 2007. Diagnosis and Classification of Diabetes Mellitus. Diabetes Care 30 (Suppl. 1):S42-S47). The independent cohort, FOS-NGT (See Table 2), was derived from the Framingham Offspring Study (FOS, (Kannel et al., Am J Epidemiol 110(3):281-290 (1979)). FOS Subjects were selected at random (balancing gender) from among all participants in the fifth FOS examination cycle (1991-1995) aged 40-49 who had no diabetes mellitus, hypertension or prior cardiovascular disease. Additional selection criteria for the FOS-NGT cohort were normal fasting glucose concentrations and normal glucose tolerance (NGT). This cohort is similar to MACS in size, gender composition and glucose tolerance, but is approximately 20 years older and differs in ancestry (Table 2).

The FOS subjects underwent an OGTT as part of the Framingham Offspring Study (Arnlov et al., Circulation 112(12):1719-1727 (2005)). After 12 hour overnight fast, subjects ingested 75 g glucose in solution. Blood samples were drawn fasting and 120 minutes after glucose ingestion.

TABLE 2
Demographic and Clinical Characteristics of Human subject Cohorts

| | Clinical Study: | | | |
|---|---|---|---|---|
| | MACS | | FOS | |
| | Cohort: | | | |
| | Glucose^{a} | Water^{b} | FOS-NGT | FOS-IGT |
| | (n = 22) | (n = 7) | (n = 25) | (n = 25) |
| Age | 23 ± 3 | 24 ± 4 | 45 ± 3 | 46 ± 3 |
| | (18-30) | (20-30) | (40-49) | (40-50) |
| Gender | 9 ♀, 13 ♂ | 3 ♀, 4 ♂ | 13 ♀, 12 ♂ | 13 ♀, 12 ♂ |
| Ancestry | Wh: 9, As: | Wh: 3, Aa: 1, | Wh: 25 | Wh: 25 |
| | 6, Un: 7 | As: 1, Un: 2 | | |
| BMI | 22.4 ± 2.1 | 22.1 ± 2.7 | 24.6 ± 3.4 | 26.8 ± 4.8 |
| | (18.3-26.9) | (17.8-26.0) | (19.0-31.5) | (18.8-41.2) |
| Fasting | 78 ± 5 | 77 ± 7 | 89 ± 6 | 100 ± 9 |
| Glucose | (71-90) | (70-86) | (76-100) | (87-115) |
| (mg/dL) | | | | |
| 120 min. | 86 ± 16 | 80 ± 9 | 88 ± 21 | 153 ± 12 |
| Glucose | (66-119) | (71-92) | (43-122) | (140-180) |
| (mg/dL) | | | | |
| Fasting | 4.6 ± 2.9 | 3.6 ± 0.7 | 4.2 ± 2.7 | 10.3 ± 8.1 |
| Insulin | (2.8-14.2) | (2.8-4.8) | (1.0-10.7) | (1.0-25.7) |
| (uIU/mL) | | | | |
| 120 min. | 18.1 ± 16.5 | 3.6 ± 1.0 | 29.7 ± 20.5 | 102.8 ± 51.8 |
| Insulin | (3.0-75.9) | (2.9-5.5) | (1.0-93.3) | (35.0-202.3) |
| (uIU/mL) | | | | |
| IGT/NGT^{c} | 0/22 | N/A | 0/25 | 25/0 |

Quantitative variables are expressed as mean ± s.d. (range).
Abbreviations:
MACS, Metabolic Abnormalities in College Students, conducted at MIT Clinical Research Center;
FOS, Framingham Offspring Study.
Ancestry abbreviations:
Wh, White;
As, Asian;
Aa, African American;
Un, Unknown
^{a}Subjects ingesting glucose (OGTT).
^{b}Subjects ingesting water (control).
^{c}Numbers of subjects in each glucose tolerance category. NGT, normal glucose tolerance; IGT, impaired glucose tolerance (American Diabetes Association 2007. Diagnosis and Classification of Diabetes Mellitus. Diabetes Care 30 (Supplement 1): S42-S47).

LC-MS/MS metabolic profiling of the OGTT time course was performed in the selected MACS subjects using the following methods.

Blood processing. Blood was drawn into EDTA coated tubes. In MACS, blood was centrifuged for 10 minutes at 6° C. and 2,000 g. Plasma samples were stored at −80° C. Sample preparation and analysis. Plasma samples were thawed gradually, and 165 μL, from each sample was mixed with 250 μL of ethanol solution (80% ethanol, 19.9% H₂O, 0.1% formic acid). After 2 hours at 4° C., the samples were centrifuged at 15,000 g for 15 minutes, and 300 μL, of the supernatant was extracted and evaporated under nitrogen. Samples were reconstituted in 60 μL, HPLC-grade water, and separated sequentially on three different HPLC columns. The columns were connected to a triple quadrupole mass spectrometer (4000 Q Trap, Applied Biosystems) operated in selected reaction monitoring mode. Each metabolite was identified by a combination of chromatographic retention time, precursor ion mass and product ion mass. Metabolite quantification was performed by integrating the peak areas of product ions using MultiQuant software (Applied Biosystem). Additional details on the analytical methodology are provided herein.

### Glucose and Insulin.

Plasma glucose concentration was measured with a hexokinase assay (MACS: Quest Diagnostics, Cambridge, Mass. FOS: Abbott Laboratories, IL). Insulin international units were determined using a radioimmunoassay (Diagnostic Product Corporation, Los Angeles, Calif.). In MACS, sodium fluoride-potassium oxalate blood tubes were used for glucose analysis, and blood tubes with no additive were used for insulin analysis. Statistical Analysis.

Statistical Tests. The significance of a change from the fasting metabolite level was calculated using the paired Wilcoxon signed-rank test. The significance of a difference between glucose and water ingestion was calculated using the unpaired Wilcoxon rank sum test. The significance of regression models was determined with an F statistic.

Significance Thresholds. Where all ˜100 detected metabolites were tested, a significance threshold of α=0.001 was used to account for multiple hypotheses testing. A threshold of α=0.05 was used elsewhere.

Linear regression of fasting insulin on 2-hour metabolite changes. The logarithm of metabolite fold change was used. The adjusted coefficient of determination (R²_{adj}) for a regression model was calculated according to the formula: R²_{adj}=1−[(n−1)/(n−m−1)]*(1−R²), where n is the number of subjects, m is the number of independent variables in the model and R² is the coefficient of determination.

Statistical analysis was performed in Matlab (The MathWorks, Inc) and in Excel (Microsoft).

HPLC

Three different HPLC systems were used sequentially. All columns were purchased from Phenomenex (Torrance, Calif.). Table 3 lists the parameters of each HPLC system:

TABLE 3
HPLC Parameters

| | System 1 | System 2 | System 3 |
|---|---|---|---|
| Mobile | ¹A: | A: | A: |
| Phase | 99.9% water | 79.75% water | 95% water |
| | 0.1% acetic acid | 20% acetonitrile | 5% acetonitrile |
| | ²B: | 0.25% ammonium | 5 mM ammonium |
| | 99.9% acetonitrile | hydroxide | acetate |
| | 0.1% acetic acid | 10 mM ammonium | B: |
| | | acetate | 95% acetonitrile |
| | | B: | 5% water |
| | | 79.75% acetonitrile | 5 mM ammonium |
| | | 20% water | acetate |
| | | 0.25% ammonium | |
| | | hydroxide | |
| | | 10 mM ammonium | |
| | | acetate | |
| Column | Luna phenyl-hexyl | Luna Amino | Synergi Polar-RP |
| | (4.6 × 50 mm, | (4.6 × 50 mm, | (4.6 × 50 mm, |
| | 5 μm) | 5 μm) | 4 μm) |
| Gradient | From 0% B and | From 100% B and | From 5% B and |
| | 1 mL/min to 90% B | 1.5 mL/min to 0% B | 1 mL/min to |
| | and 2 mL/min in | and 2.5 mL/min in | 95% B and 2 mL/ |
| | 0.7 minutes | 1.6 minutes | min in 2.65 |
| | | minutes | |
| Injection | 5 μL | 10 μL | 10 μL |
| Volume | | | |

¹A: Aqueous phase
²B: Organic phase.

Mass Spectrometry

A Turbo electrospray ionization source was used. The ion spray potentials were 5,000 volt in the positive mode and 4,200 volt in the negative mode. Zero air was used for the nebulizer and bath gases, and N₂ was used for the curtain and collision gases. The gas pressures used were 50 psi for the nebulizer gas, 60 psi for the bath gas, 20 psi for the curtain gas and 7 psi for the collision gas. The bath gas temperature was 400° C. Table 4 lists the mass spectrometry parameters, HPLC system and standard source information for all metabolites discussed in the text.

TABLE 4
Mass Spectrometry Parameters, HPLC System and Standard Sources

| | | | | | | | | Standard |
|---|---|---|---|---|---|---|---|---|
| | | | | | | | Standard | Catalog |
| Metabolite Name | ¹Q1 | ²Q3 | ³DP | ⁴CE | ⁵IP | ⁶HPLC | Source | Number |
| Alanine | 90.0 | 44.0 | 25 | 15 | + | 1 | ⁷Sigma | A-7627 |
| Arginine | 175.1 | 70.0 | 25 | 30 | + | 1 | Sigma | A-5131 |
| β-hydroxybutyrate | 103.0 | 59.0 | −40 | −15 | − | 3 | Sigma | 54920 |
| Citrulline | 174.1 | 131.0 | −50 | −15 | − | 3 | Sigma | 27510 |
| Glucose | 179.1 | 89.0 | −50 | −15 | − | 2 | Sigma | 49159 |
| Glycerol | 93.0 | 57.0 | 20 | 21 | + | 1 | Shelton | IB15762 |
| | | | | | | | Scientific- | |
| | | | | | | | IBI | |
| Glycochenodeoxycholic | 448.3 | 74.0 | −80 | −60 | − | 3 | Sigma | G0759 |
| acid | | | | | | | | |
| Glycocholic acid | 464.3 | 74.0 | −30 | −60 | − | 3 | Sigma | G2878 |
| Hippuric acid | 178.1 | 134.0 | −50 | −16 | − | 3 | Sigma | 112003 |
| Histidine | 156.1 | 110.0 | 25 | 23 | + | 1 | Sigma | H-8125 |
| Hypoxanthine | 135.0 | 92.0 | −50 | −23 | − | 3 | Sigma | 56700 |
| Isoleucine | 132.1 | 86.0 | 50 | 20 | + | 1 | Sigma | 1-2752 |
| Lactate | 89.0 | 43.0 | −40 | −20 | − | 2 | Sigma | 69771 |
| Leucine | 132.1 | 86.0 | 50 | 20 | + | 1 | Sigma | L-8000 |
| Lysine | 147.1 | 84.0 | 25 | 25 | + | 1 | Sigma | G-3126 |
| Malate | 133.0 | 115.0 | −40 | −20 | − | 3 | Sigma | M-0750 |
| Methionine | 150.1 | 61.0 | 40 | 30 | + | 1 | Sigma | M-9625 |
| Ornithine | 133.1 | 70.0 | 40 | 30 | + | 1 | Sigma | 75480 |
| Phenylalanine | 166.1 | 120.0 | 50 | 17 | + | 1 | Sigma | P-2126 |
| Pyruvate | 87.0 | 43.0 | −30 | −12 | − | 3 | Sigma | 107360 |
| Taurochenodeoxycholic | 498.3 | 80.0 | −90 | −90 | − | 3 | Sigma | 86335 |
| acid | | | | | | | | |
| Tyrosine | 182.1 | 136.5 | 25 | 17 | + | 1 | Sigma | T-3754 |
| Valine | 118.1 | 72.0 | 25 | 20 | + | 1 | Sigma | V-0500 |

¹Q1: precursor ion mass, in daltons (Da)
²Q3: product ion mass, in daltons (Da)
³DP: De-clustering Potential, in electronvolts (eV)
⁴CE: Collision Energy, in electronvolts (eV)
⁵IP: Ionization Polarity
⁶HPLC: the HPLC system in which the metabolite was measured. See the HPLC section above for the parameters of each system.
⁷Sigma: Sigma-Aldrich Co.

Metabolite Interferences

The HPLC-MS/MS method was unable to distinguish a few metabolites in the table above from other tested metabolites, due to a combination of isobaric overlap and insufficient chromatographic resolution. Leucine and isoleucine were indistinguishable, and are therefore always mentioned together in the text. In the other instances of sets of indistinguishable metabolites, one of the metabolites in the set was likely to be present in the samples in much higher concentrations than the rest of the set, based on reported concentrations in human plasma and given the nature of an oral glucose tolerance test. In these instances, it was assumed that the effect of the non-prevalent metabolites on the measurement was negligible, and only the prevalent metabolite was mentioned in the text. The sets of indistinguishable metabolites are listed below, with the prevalent metabolite first: {glucose, galactose, fructose}, {β-hydroxybutyrate, malonate}, and {valine, guanidinoacetate}.

Results

Out of the 191 metabolites monitored as described above, 97 were detected in at least 80% of subjects in all time points (FIG. 1B). The levels of 21 metabolites changed significantly (p<0.001) from the fasting levels and were also significantly (p<0.05) different when compared to the response to water (FIG. 1C). These 21 significantly altered metabolites span pathways previously studied in the context of glucose homeostasis, as well as some never linked to this program.

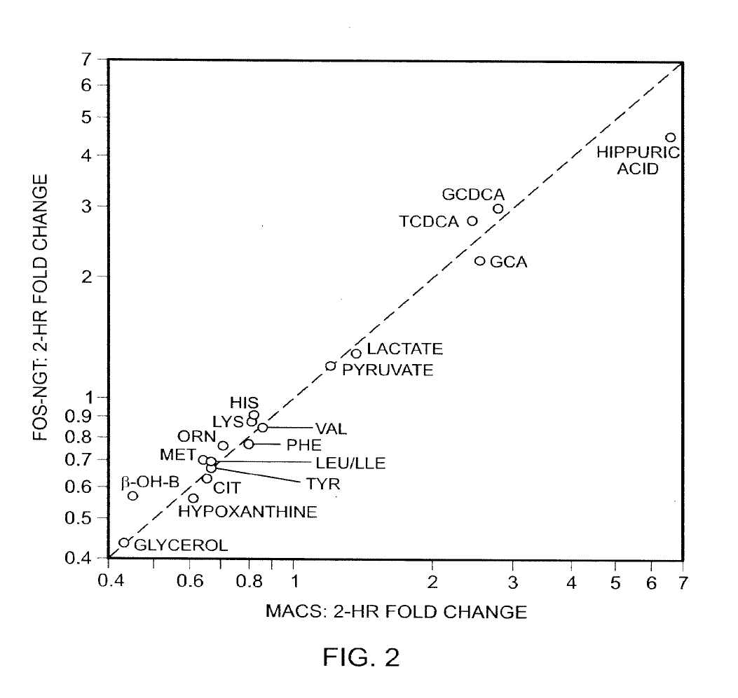

Of the 21 metabolites displaying significant change in MACS at any time-point during OGTT (FIG. 1C), the levels of 20 (glucose excluded) remained significantly (p<0.05) altered at the 2-hour time point. 18 of these 20 metabolite changes replicated significantly (p<0.05) and in the same direction in FOS-NGT (FIG. 2). The remaining two metabolites, malate and arginine, fell below the selected significance threshold. Thus 18 plasma metabolites that exhibit highly reproducible and likely robust responses to glucose ingestion in healthy subjects have been identified.

### Example 2

### Metabolic Profiling Reveals Novel Biochemical Changes During OGTT

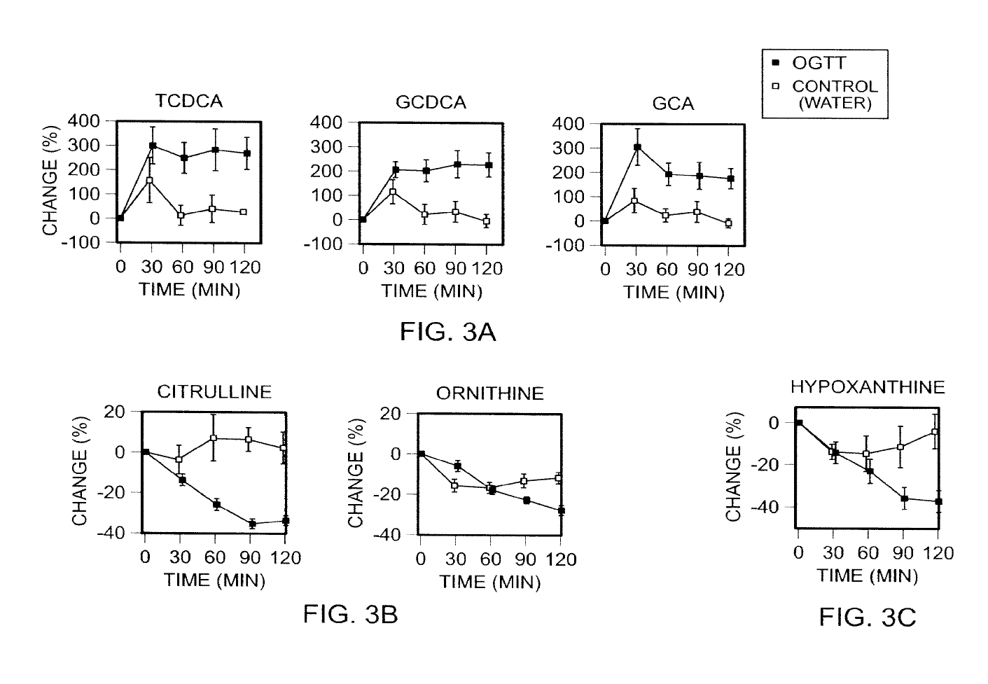

The systematic profiling approach has enabled the identification of a number of plasma metabolites, not previously associated with glucose homeostasis, that change reproducibly in response to an oral glucose challenge. Perhaps most striking was the observed changes in bile acids. The levels of three bile acids—glycocholic acid (GCA), glycochenodeoxycholic acid (GCDCA) and taurochenodeoxycholic acid (TCDCA)—more than doubled during the first 30 minutes after glucose ingestion (FIG. 3A). The levels of these bile acids remained elevated for the entire two hours. Water ingestion produced a smaller increase in bile acids which did not persist beyond the 30 minutes time point. All three compounds are primary bile acids conjugated to glycine or taurine.

Other novel changes were also observed. The levels of citrulline and ornithine, two non-proteinogenic amino acids which participate in hepatic urea synthesis, decreased by 35% and 29% respectively during the 2-hour test (FIG. 3B). The levels of hypoxanthine, a purine base generated from degradation of adenine and guanine nucleotides, decreased in MACS by 39% within two hours of glucose ingestion (FIG. 3C), and this pattern was replicated in FOS-NGT. Xanthine, a purine base generated from hypoxanthine by oxidation, also decreased in both cohorts (MACS: 9%, p<0.05; FOS-NGT: 41%, p<10⁻⁴). Interestingly, hippuric acid increased by over 1000% during the first 30 minutes and decreased gradually thereafter. Most likely this response is not related to glucose, but rather reflects the presence of the preservative benzoic acid, a precursor of hippuric acid (Kubota and Ishizaki, Eur J Clin Pharmacol 41(4):363-368 (1991)), in the glucose solution used for OGTT described herein.

### Example 3

### Changes in Plasma Metabolites Span Four Arms of Insulin Action

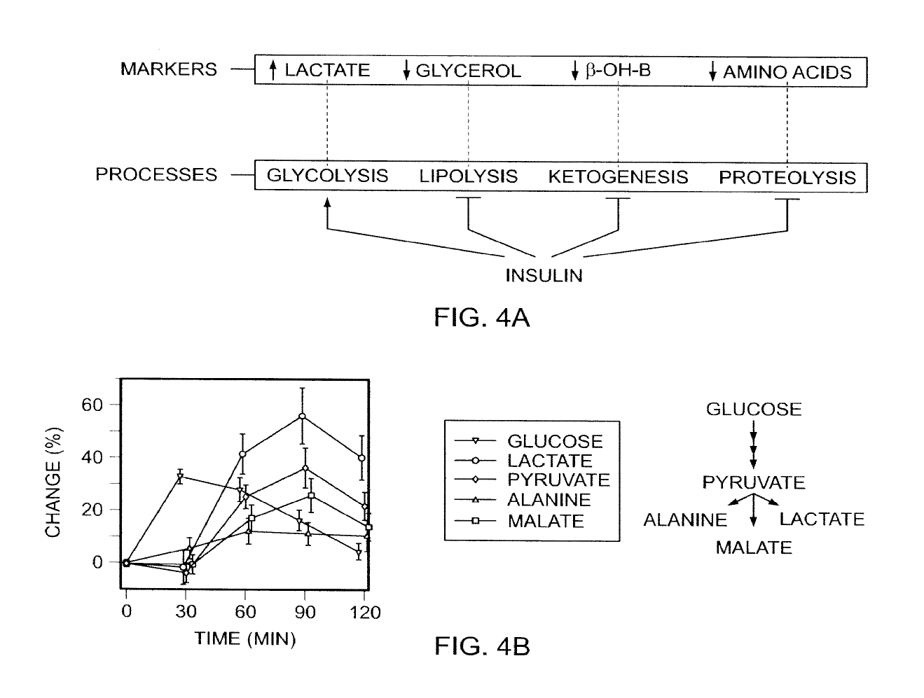

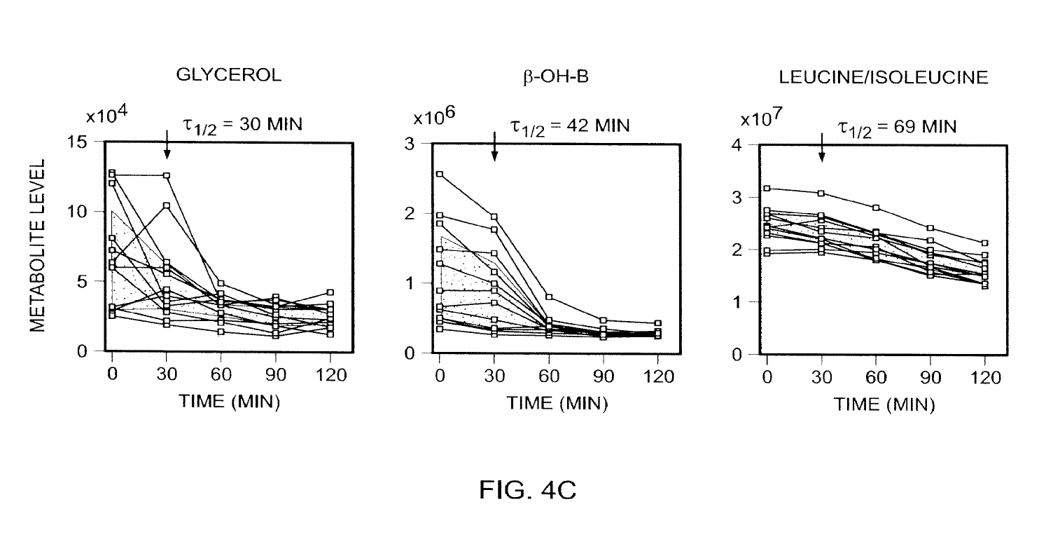

Much of the biochemical response to glucose ingestion, which were studied in an unbiased way, can be attributed to the action of insulin. Specifically, metabolite changes corresponding to the stimulation of glucose metabolism and to the suppression of lipolysis, ketogenesis and proteolysis, were detected, all of which are known to be elicited by insulin (FIG. 4A).

The methods described herein captured the temporal relationship between glucose and intermediates of glycolysis (FIG. 4B). Specifically, the increase of pyruvate, lactate and alanine occurred between 30 and 60 minutes, lagging ˜30 minutes behind the glucose rise, consistent with previous reports (Kelley et al., J Clin Invest 81(5):1563-1571 (1988)). Interestingly, the kinetics of malate, an intermediate in the Krebs cycle, closely resembled the kinetics of lactate and pyruvate. To the present inventors' knowledge this observation has not been previously reported, and suggests that part of the pyruvate formed through glycolysis was carboxylated to generate malate, causing an elevation of plasma malate levels.

To gain insight into the kinetics of insulin action, temporal patterns for metabolites indicative of the suppression of fat and protein catabolism were compared. The levels of glycerol, β-hydroxybutyrate, and multiple amino acids all declined after glucose ingestion, but the kinetic pattern of glycerol and β-hydroxybutyrate was remarkably different from amino acids (FIG. 4C). Over the two hours, the decrease of glycerol and β-hydroxybutyrate levels was 57% and 55% respectively, while the drop in amino acids was moderate (between 14-36%). The branched-chain amino acids leucine/isoleucine (indistinguishable by our method), for example, decreased 33%. Interestingly, the median time to reach half-maximal decrease was also greater for the amino acids (50-72 minutes) than for β-hydroxybutyrate (42 minutes) and glycerol (30 minutes). Moreover, the inter-subject variance in metabolite levels shrunk dramatically over the two hours in glycerol and β-hydroxybutyrate (84% and 95% reduction of inter-quartile range, respectively), while in amino acids the maximal reduction was 53%. These findings suggest that the suppression of lipolysis and ketogenesis may be more sensitive to the action of insulin compared to suppression of protein catabolism.

### Example 4

### Metabolite Markers Reflect the Individuality of Insulin Sensitivity

Metabolites that exhibit robust 2-hour changes were further evaluated to determine whether they might be useful in understanding insulin sensitivity. Insulin sensitivity is traditionally defined as the ability of insulin to promote the uptake of glucose into peripheral tissues such as skeletal muscle and fat. A decline of insulin sensitivity is one of the earliest signs of type 2 diabetes mellitus (T2DM). This decline is often manifest as elevated levels of fasting insulin, and a strong correlation exists between fasting insulin and direct measurements of insulin sensitivity (Hanson et al., Am J Epidemiol 151(2):190-198 (2000)). Considering that several metabolic processes taking place in response to glucose ingestion are mediated by insulin, it was hypothesized that insulin sensitivity could be reflected not only by glucose, but also by the OGTT-response of multiple other metabolites.

The following experiments were performed to determine if the OGTT-response of the 18 metabolites that demonstrated robust 2-hour changes could be predictive of fasting insulin. Because the initial studies described above were focused on normal, healthy subjects spanning a narrow range of fasting insulin levels, a third analysis was performed on a cohort of subjects with impaired glucose tolerance from the Framingham Offspring Study (FOS-IGT), who spanned a broader range of fasting insulin concentrations (Table 2). Additional selection criterion for the FOS-IGT cohort was impaired glucose tolerance (2-hour glucose concentration between 140 and 199 mg/dL). The metabolites were evaluated using the same methods as described above for the MACS study, but in the FOS study, the blood samples were centrifuged for 30 minutes at 4° C. and 1,950 g. First, to systematically evaluate the relationship between subject metabolite changes and fasting insulin, linear regression of the fasting insulin concentration was performed on each of the 18 metabolite changes. Out of the 18 metabolites, six showed a statistically significant (p<0.05) correlation to fasting insulin, and included lactate, β-hydroxybutyrate, amino acids (leucine/isoleucine, valine and methionine) and a bile acid (GCDCA). Taken together with glycerol, which scored (p=0.07) slightly below the significance threshold, the response of four distinct insulin action markers correlated with fasting insulin (FIG. 5A). Subjects with high fasting insulin exhibited blunted metabolite response in all four markers. These findings suggest that resistance to the action of insulin on the metabolism of glucose, fat, and protein is reflected by the metabolite response to OGTT.

Next, experiments were performed to determine whether a combination of metabolite changes might be more predictive of insulin sensitivity than are subject metabolites. Forward stepwise linear regression was used to discover an optimal linear model (adjusted for the number of explanatory variables) for predicting fasting insulin levels. The top regression model that was identified consisted of a combination of Leu/Ile and glycerol (R²_{adj}=0.54, p=0.0001). In this bivariate model, the independent contribution from each of Leu/Ile and glycerol was significant (p=8×10⁻⁵, p=4×10⁻³ respectively). These two predictors were not correlated with each other (p=0.6). Adjusting for the number of predictor variables, the Leu/Ile-glycerol model predicted fasting insulin levels better than any individual metabolite change (FIG. 5B). BMI, which is known to be a strong predictor of fasting insulin, was less predictive than the bivariate model. Notably, the explanatory power of Leu/Ile and glycerol was significant even after controlling for BMI (p=2×10⁻³, p=3×10⁻³ respectively). A graphical representation of the Leu/Ile-glycerol model (FIG. 5C) demonstrates that some subjects with high fasting insulin exhibit a blunted decline in glycerol, while others exhibit a blunted decline in Leu/Ile.

### Example 5

### Metabolite Markers Reflect the Propensity to Develop Type 2 Diabetes

It is possible that alterations in metabolite levels could presage the onset of overt DM, and thus represent useful biomarkers. However, this hypothesis has yet to be examined in a prospective, longitudinal study. Therefore, metabolomic profiling was performed in participants from the community-based Framingham Heart Study, with the goal of identifying novel predictors of future DM.

The Framingham Offspring Study was initiated in 1971, when 5,124 offspring (and their spouses) of the original Framingham Heart Study participants were enrolled into a longitudinal cohort study (Kannel et al., Am J. Epidemiol. 110:281-290 (1979)). Participants in this cohort are examined every 4 years. At each quadrennial Framingham visit, participants underwent a physician-administered physical examination and medical history, and routine laboratory tests that included fasting glucose. The 5^{th} examination of this cohort took place in 1991 through 1995, and was chosen as the baseline examination. At this examination, participants were administered a 2-hour oral glucose tolerance test after a 12-hour overnight fast, using 75 grams of glucose in solution.

The presence of DM, ascertained at every visit, was defined by a fasting glucose 126 mg/dl or use of insulin or hypoglycemic medications. Individuals with a 2-hour glucose 200 mg/dl on the oral glucose tolerance test administered at the baseline examination were also said to have DM and excluded from the investigation.

A nested case-control design was used to evaluate metabolomic predictors of DM development. A total of 193 individuals developed DM after the baseline examination, over a 12-year follow-up period (e.g. 3 follow-up examinations). These individuals were designated as cases. Propensity matching was used to identify an equal number of controls. Logistic regression models were used to generate the propensity scores. For these models, DM was the outcome variable, and the following variables were used as covariates: age, body mass index, fasting glucose, and hypertension (defined as blood pressure 140/90 or use of anti-hypertensive therapy). Selection of clinical covariates was based on prior reports (Wilson et al., Arch. Intern Med. 167(10):1068-1074 (2007)). Six separate logistic regression models were estimated, one for each follow-up examination and gender. Each case was matched to the control with the closest exam- and gender-specific propensity score, provided the difference in propensity scores was <0.10. A control could only be used once. Using this approach, a propensity-matched control was identified for all but 4 cases (2 women). Thus, the final sample included 189 cases and 189 controls.

The association between the levels of 60 metabolites (pre- and post-oral glucose loading) and incident DM was examined. Log transformation was applied to metabolite levels (intensity units), in order to correct for heteroscedasticity of the case-control differences in the untransformed data. Baseline metabolite levels were compared in cases (individuals who went on to develop DM) versus controls (individuals who did not develop DM) using paired t-tests for the 46 metabolites with <5% missing data. The remaining 14 metabolites had undetectable levels in 5% of samples. For these metabolites, McNemar's tests were used in place of paired t-tests, to compare the proportion of detectable values in cases versus controls.

Because individuals also underwent 2-hour oral glucose tolerance tests, we performed analyses to assess whether the excursion in metabolite levels during the oral glucose tolerance test was associated with incident DM. The 2-hour metabolite level was regressed on the baseline metabolite level, case status, and an interaction term (case status×baseline metabolite level). For the 14 metabolites with a large proportion of levels below the detection limit, binary variables were used in place of continuous ones.

For selected metabolites, conditional logistic regression analyses were performed to estimate the relative risk of DM at different metabolite values. Conditional logistic regression was used rather than conventional logistic regression in order to account for the matched pairs. For these analyses, the metabolites were treated as continuous and as categorical variables. The distributions were standardized to have a standard deviation (SD) of 1. Sex-specific quartiles were used based on the distribution of the metabolites in the control sample. Regressions were adjusted for age, sex, body mass index, and fasting glucose. In secondary analyses, fasting insulin, dietary protein, dietary amino acids, and total caloric intake were also adjusted for.

Characteristics of the study sample are shown in Table 5. As expected from the matching process, there were no significant differences between cases and controls with respect to the following clinical risk factors for DM: age, gender, body mass index, or fasting glucose.

TABLE 5
Characteristics of the study sample

| | | | Individuals |
|---|---|---|---|
| | | Individuals who | who did not |
| | Whole | developed | develop |
| | sample | diabetes | diabetes |
| | (n = 378) | (n = 189) | (n = 189) |
| Baseline age, years | 57 ± 9 | 56 ± 9 | 57 ± 9 |
| Women, % | 42% | 42% | 42% |
| Baseline body mass | 30.3 ± 5.3 | 30.5 ± 5.0 | 30.0 ± 5.5 |
| index, kg/m² | | | |
| Baseline hypertension, % | 53% | 53% | 53% |
| Baseline fasting glucose, | 104.8 ± 8.8 | 104.7 ± 9.1 | 104.9 ± 8.5 |
| mg/dl | | | |

Values in Table 5 are mean ± SD, or percentage.

Results of analyses comparing those who went on to develop DM (cases) and those who did not (controls) are shown in Table 6, for selected metabolites. Seven metabolites had p-values of 0.005 or smaller for the baseline differences between those who did and did not develop DM, and 5 metabolites had p-values of 0.001 or smaller. Three of these metabolites were the branched chain amino acids: leucine (p=0.0005), isoleucine (p=0.0001), and valine (p=0.001). Two were aromatic amino acids: phenylalanine (p<0.0001) and tyrosine (p<0.0001). There was no evidence of an interaction between case status and the 2-hour OGTT results for these metabolites, suggesting that metabolite concentrations after OGTT did not add predictive information to the baseline concentrations. In additional analyses stratified by duration of follow-up, there was no evidence of an interaction between follow-up year and case-control difference for any of top metabolites (P>0.10 for all tests of interaction). Thus, the metabolites appeared to retain their predictive value for DM as long as 12 years after the baseline examination.

TABLE 6
Comparison of baseline metabolite levels in individuals with and without
incident diabetes

| | Type of | |
|---|---|---|
| Metabolite | variable | P-value |
| phenylalanine | Continuous | <.0001 |
| tyrosine | Continuous | <.0001 |
| isoleucine | Continuous | 0.0001 |
| leucine | Continuous | 0.0005 |
| valine | Continuous | 0.001 |
| ornithine | Continuous | 0.002 |
| tryptophan | Continuous | 0.003 |

Conditional logistic regression models were performed to assess the association between baseline metabolite levels and future DM, after adjustment for age, sex, body mass index, and fasting glucose (Table 7).

TABLE 7
Relation of baseline amino acid levels to risk of future DM

| | | | | | |
|---|---|---|---|---|---|
| Model | Isoleucine | Leucine | Valine | Tyrosine | Phenylalanine |

Metabolite as continuous variable

| | | | | | |
|---|---|---|---|---|---|
| Per SD | 1.70 | 1.62 | 1.57 | 1.85 | 2.02 |
| increment | (1.27-2.28) | (1.20-2.17) | (1.17-2.09) | (1.35-2.55) | (1.40-2.92) |
| P | 0.0004 | 0.001 | 0.002 | 0.0001 | 0.0002 |

Metabolite as categorical variable

| | | | | | |
|---|---|---|---|---|---|
| First | 1.0 | 1.0 | 1.0 | 1.0 | 1.0 |
| quartile | (referent) | (referent) | (referent) | (referent) | (referent) |
| Second | 1.54 | 1.81 | 1.29 | 1.96 | 1.25 |
| quartile | (0.81-2.94) | (0.94-3.49) | (0.70-2.35) | (1.01-3.79) | (0.69-2.29) |
| Third | 2.59 | 4.47 | 1.424 | 2.69 | 1.83 |
| quartile | (1.28-5.24) | (2.07-9.65) | (0.74-2.75) | (1.33-5.43) | (0.94-3.58) |
| Fourth | 3.61 | 3.96 | 2.66 | 2.58 | 1.98 |
| quartile | (1.59-8.20) | (1.76-8.91) | (1.21-5.87) | (1.21-5.49) | (0.90-4.36) |
| P for trend | 0.001 | 0.0005 | 0.02 | 0.01 | 0.08 |

Values are hazards ratios (95% confidence intervals) for DM, from conditional logistic regressions.
All models are adjusted for age, sex, body mass index, and fasting glucose.

For the top 5 metabolite results, each SD increment in log marker was associated with a 50% to 100% increased risk of future DM (p=0.0001 to 0.002). Individuals in the top quartile of metabolite values at baseline had 2-fold (for phenylalanine) to 4-fold (for leucine) risks of developing DM over the 12-year follow-up period, compared with those with metabolite values in the lowest quartile. Results were similar when models were further adjusted for baseline insulin, dietary protein intake, dietary amino acids, and total caloric intake.

These results demonstrate that these metabolites can be used to predict a subject's risk of developing diabetes mellitus in the future.

### ADDITIONAL REFERENCES

Fukagawa et al., J Clin Invest 76(6):2306-2311.
Kaya et al., Metabolism 55(1):103-107.
Mateos et al., J Clin Invest 79(3):847-852.
Nurjhan et al., Diabetes 35(12):1326-1331.
Sutton et al., Metabolism 29(3):254-260.
Yamaoka et al., J Biol Chem 272(28):17719-17725.

### Other Embodiments

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only. All references described herein are incorporated by reference for the purposes described herein.

Moreover, this invention is not limited in its application to the details of construction and the arrangement of components set forth in the disclosed description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A method for determining the risk of developing diabetes in a subject, the method comprising:
determining a level of two or more metabolic biomarkers in the sample, wherein the metabolic biomarkers are selected from the group consisting of isoleucine, phenylalanine, tyrosine, valine, leucine, tryptophan, and ornithine; and
comparing the levels of the metabolic biomarkers with reference levels of the same biomarkers,
wherein the presence of levels of the metabolic biomarkers that are higher than the reference levels indicates an increased risk of developing diabetes in the subject.

2. The method of claim 1, comprising determining levels of isoleucine and one or more of phenylalanine, tyrosine, valine, leucine, tryptophan, or ornithine.

3. The method of claim 1, comprising determining levels of isoleucine, phenylalanine, tyrosine, valine, leucine, tryptophan, and ornithine.

4. The method of claim 1, further comprising determining a level of an additional biomarker selected from the group consisting of glycerol, lactate, and β-hydroxybutyrate.

5. The method of claim 1, further comprising determining a level of an additional biomarker selected from the group consisting of citrulline, glycochenodeoxycholic acid, glycocholic acid, hippuric acid, histidine, hypoxanthine, lysine, methionine, pyruvate, and taurochenodeoxycholic acid.

6. The method of claim 1, wherein the subject has normal glucose tolerance.

7. The method of claim 1, wherein the sample comprises serum from the subject.

8. The method of claim 1, further comprising selecting a treatment for the subject based on the comparison of the levels of the metabolic biomarkers with the reference levels.

9. The method of claim 8, further comprising administering the selected treatment to the subject.

10. The method of claim 8, wherein the treatment is administering to the subject an effective amount of at least one anti-diabetes compound.

11. The method of claim 1, wherein the biological sample is obtained from the subject following a fast.

12. The method of claim 10, wherein the fast was for between 6 and 16 hours.

13. The method of claim 1, further comprising assessing one or both of glucose and insulin levels in the subject.

14. The method of claim 1, wherein the subject has at least one risk factor for diabetes.

15. The method of claim 1, wherein the levels of the biomarkers are determined using a mass spectrometer.

16. A kit for determining the presence or risk of a glucose related metabolic disorder in a subject, the kit comprising:
reagents suitable for determining levels of a plurality of biomarkers in a test sample, wherein the plurality of biomarkers comprises two or more of isoleucine, phenylalanine, tyrosine, valine, leucine, tryptophan, and ornithine; and
optionally one or more control samples comprising predetermined levels of the same biomarkers, wherein a comparison of the levels of the biomarkers in the test sample with the levels in the control samples indicates the presence of risk of a glucose related metabolic disorder in the subject.
